SEQUENCE LISTING (6 sequences)

SEQ ID NO: 1 - DNA, 19 bp, Artificial Sequence
  primer
gtcctcgtcctcctaccgc

SEQ ID NO: 2 - DNA, 20 bp, Artificial Sequence
  primer
cgggctcctgcaggactcgg

SEQ ID NO: 3 - DNA, 20 bp, Artificial Sequence
  primer
atgttcggcggcccgggcac

SEQ ID NO: 4 - DNA, 21 bp, Artificial Sequence
  primer
aggagccgcaccccgggcacg

SEQ ID NO: 5 - DNA, 20 bp, Artificial Sequence
  primer
gaggggaccctctttcctaa

SEQ ID NO: 6 - DNA, 20 bp, Artificial Sequence
  primer
ggtggacgtagtcacgtagc

(19) United States
(12) Patent Application Publication
(10) Pub. No.: US 2025/0041311 A1
(43) Pub. Date: Feb. 6, 2025

(54) Compound For Treating or Preventing Vimentin-Mediated Diseases

(71) Applicant: Aluda Pharmaceuticals, Inc., Union City, CA (US)

(72) Inventors: Jianping Wu, Chuzhou City (CN); Lian Mo, Union City, CA (US); Ruihuan Chen, Union City, CA (US)

(21) Appl. No.: 18/275,603

(22) PCT Filed: Jan. 27, 2022
(86) PCT No.: PCT/CN2022/074275

§ 371 Date: Aug. 12, 2024

(30) Foreign Application Priority Data
Feb. 3, 2021 (CN) 202110148083.9
Feb. 3, 2021 (CN) 202110148251.4

(51) Int. Cl. A61K 31/5377

(52) U.S. Cl. CPC A61K 31/5377

(57) ABSTRACT

Provided in the present invention is a compound for treating or preventing vimentin-mediated diseases. The compound of the present invention is a s-triazine derivative, as shown in the following formula A, or a pharmaceutically acceptable carrier, prodrug, enantiomer, diastereomer, tautomer or solvate thereof, wherein R₁, R₂ and Z are as defined herein. The present invention further comprises the corresponding pharmaceutical use, and a method for treating and preventing diseases.

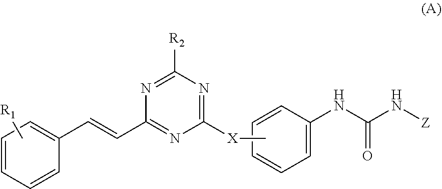

### TECHNICAL FIELD

The present invention relates to compounds for the treatment or prevention of diseases where vimentin is implicated.

### BACKGROUND TECHNIQUE

Vimentin is an intermediate filament protein that plays an important role in normal cellular processes, such as cell endocytosis, exocytosis, and intracellular material transportation, and in the onset and progression of various diseases, such as infectious diseases and cancer (Danielsson, F. et al., Vimentin Diversity in Health and Disease, Cells 7:147; 2018).

There are a variety of pathogens that infect humans, including bacteria and viruses. Finding effective drugs against specific pathogens is a routine drug development strategy. This type of drug is only effective against a specific pathogen but not against a mutated version of that pathogen or a different pathogen. Unlike direct pathogen-fighting drugs, host-directed drug can have broad-spectrum anti-pathogenic effects, because many types of pathogens spread by hijacking cellular processes of the host. To successfully infect a cell, pathogens must first enter host cells, then move to a suitable site within the cells to replicate, and finally release the progeny pathogens to complete the infection cycle. Many pathogens hijack the same cellular processes, such as through endocytosis to enter the host cells, through endosomal trafficking to move to appropriate intracellular locations for replication, and through the exosomal pathway to release progeny pathogens.

There are many pathogens that use the above cellular processes to infect host cells, including but not limited to coronavirus, HIV, influenza virus, hepatitis B virus, hepatitis C virus, human papilloma virus, Ebola virus, dengue virus, Escherichia coli, Salmonella enteritidis, Anaplasma phagocytophilum, Chlamydia trachomatis, Streptococcus pyogenes, Mycobacterium tuberculosis, Mycobacterium avium and Propionibacterium acnes, etc.

Cancer is a completely different disease from bacterial and viral infections. Like pathogens, however, cancer cells use these cellular processes for their growth and spread. Cancer cells rely on endocytosis to efficiently uptake nutrients (Commisso, C. et al., Macropinocytosis of protein is an amino acid supply route in Ras-transformed cells, Nature 497, 633-637; 2013), and rely on releasing exosomes to create a microenvironment suitable for the growth and metastasis of cancer cells (Pegtel, D. M., Gould, S. J., Exosomes, Annu. Rev. Biochem. 88, 487-514, 2019; Kalluri, R., LeBleu, V. S., The biology, function, and biomedical applications of exosomes, Science 367, eaau6977, 2020).

Intervening the cellular processes, including endocytosis, endosomal trafficking and exosomal release, can have a broad therapeutic effect against different pathogens (regardless of their specific types, variants and mutants) and against different cancers (regardless of their cell types).

Vimentin is utilized by various bacteria (Mak et al., Vimentin in bacterial infections. Cells. 5(2):18, 2016) as well as viruses (Zhang, et al., The diverse roles and dynamic rearrangement of vimentin during viral infection, J Cell Sci. 134: jcs250597, 2021) to achieve their productive infection. These pathogens often use vimentin to attach to host cells (Du, et al., Cell Surface Vimentin Is an Attachment Receptor for Enterovirus 71, J Virol. 88: 5816-5833, 2014), to enter the cells (Schäfer, et al., Vimentin Modulates Infectious Internalization of Human Papillomavirus 16 Pseudoviruses, J Virol. 91(16):e00307-17, 2017), to traverse inside the cells (Wu, et al., Vimentin plays a role in the release of the influenza A viral genome from endosomes, Virology. 497:41-52, 2016), to replicate (Turkki, et al., Human Enterovirus Group B Viruses Rely on Vimentin Dynamics for Efficient Processing of Viral Nonstructural Proteins, J Virol. 94(2):e01393-19, 2020), and eventually to release from the cells (Bhattacharya, et al., Interaction between Bluetongue virus outer capsid protein VP2 and vimentin is necessary for virus egress, Virol J. 4:7, 2007).

Vimentin is a potential target for cancer therapy (Strouhalova, K. et al., Vimentin Intermediate Filaments as Potential Target for Cancer Treatment, Cancers (Basel) 12, 184, 2020), because vimentin plays a key role in several cellular processes that cancer cells rely on, for example, epithelial-to-mesenchymal transition (EMT) (Liu, C. Y et al., Vimentin contributes to epithelial-mesenchymal transition cancer cell mechanics by mediating cytoskeletal organization and focal adhesion maturation, Oncotarget 6, 15966-15983, 2015), cell migration and invasion (Sharma. P. et al., Intermediate Filaments as Effectors of Cancer Development and Metastasis: A Focus on Keratins, Vimentin, and Nestin, Cells 8:497, 2019). These are common features of all solid tumors and are not limited to specific tumor types.

An imbalanced immune system and/or abnormal inflammatory responses are major components of various human diseases. Individuals with these diseases often exhibit inadequate numbers and/or abnormal functions of regulatory T cells (Dominguez-Villar, et al. Regulatory T cells in autoimmune disease. Nat. Immunol. 19:665-673, 2018).

Common medical problems caused by inflammation and/or disturbances in Treg cell homeostasis include, but are not limited to: multiple sclerosis (MS), a classic autoimmune and inflammatory disease in which the immune system directly targets the central nervous system; inflammatory Bowel disease (IBD), a chronic gastrointestinal inflammatory disorder including Crohn's disease and ulcerative colitis; systemic lupus erythematosus (SLE) is a systemic autoimmune disease in which inflammation affects many different organs and systems; type 1 diabetes (T1D), a manifest of pancreatic beta cells destruction by autoimmunity; psoriasis, a chronic autoimmune disease that causes the rapid buildup of skin cells; graft versus host disease (GvHD), an immune-mediated condition occurs after bone marrow or solid organ transplantation; myasthenia gravis (MG), a long-term neuromuscular disease that causes varying degrees of skeletal muscle weakness; viral infections, such as COVID-19 in which a virus-induced overreaction of the immune system leads to tissue damage and organ failure. Other related diseases include arthritis, scleroderma, dermatomyositis, vasculitis, neuritis, autoimmune hemolytic anemia, pernicious anemia with chronic atrophic gastritis, pulmonary hemorrhage nephritic syndrome, primary biliary cirrhosis, thyroid autoimmune disease, pemphigus, Sjorgen's syndrome, fibrosis, atherosclerosis, chronic kidney disease, osteoporosis, allergies, fibromyalgia, neurodegeneration and cancer, etc.

Vimentin, as an intermediate filament protein, restrains the function of Treg cells (McDonald-Hyman, C. et al. The Vimentin Intermediate Filament Network Restrains Regulatory T Cell Suppression of Graft-Versus-Host Disease. J. Clin. Invest. 128:4604-4621, 2018). The vimentin depletion activates Treg cells, inactivates the NLRP3 inflammasome (Dos Santos, G. et al., Vimentin Regulates Activation of the NLRP3 Inflammasome. Nat. Commun. 6:6574, 2015), reduces inflammation and protects animals from tissue injury (Surolia, R. et al., Vimentin intermediate filament assembly regulates fibroblast invasion in fibrogenic lung injury. JCI Insight. 4(7):e123253, 2019).

### DESCRIPTION OF THE INVENTION

The first aspect of the present invention provides the s-triazine derivative represented by the following formula A, or its pharmaceutically acceptable carrier, prodrug, enantiomer, diastereomer, tautomer or solvate in the preparation of medicines for the treatment or prevention of diseases where vimentin is implicated, including uses in the preparation of medicines for the treatment or prevention of diseases related to cell endocytosis, exocytosis and endosomal trafficking, and in the preparation of medicines for the treatment or prevention of diseases associated with inadequate number and/or function of regulatory T cells:

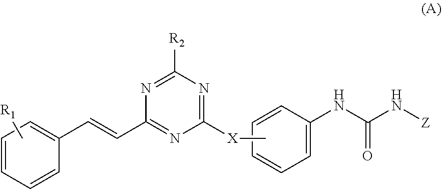

in the formula A:
R₁ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl or aminomethyl;
R₂ is —NR₄R₅, wherein R₄ and R₅ are independently selected from a group consisting of hydrogen, C₁-C₆ alkyl, and C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, hydroxyl, C₁-C₆ alkyl, or C₁-C₆ haloalkyl;
Z is aryl or heteroaryl optionally substituted by 1 to 3 R₃; preferably, said aryl is 6- to 14-membered aryl, such as phenyl or naphthyl; said heteroaryl is 5-10 membered heteroaryl, preferably nitrogen-containing heteroaryl, including but not limited to imidazolyl, pyrazolyl, pyridyl, pyrimidinyl, pyrazinyl, pyridazinyl, pyrrolyl, triazolyl and tetrazolyl; preferably, Z is phenyl or pyridyl optionally substituted by 1 or 2 R₃;
R₃ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl, aminomethyl or —CORₐ;
Wherein Rₐ is OH or NR₇R₈, wherein R₇ and R₈ are independently selected from hydrogen, C₁-C₆ alkyl optionally substituted with one or more substituents selected from halogen or NR₉R₁₀, and C₁-C₆ alkyl substituted with 3-(C₂-C₆ alkynyl)-3H-diaziridinyl; or R₇, R₈ and the nitrogen atom bonded to them form a 4 to 6-membered heterocyclic ring containing optionally an additional heteroatom selected from N, O and S, and optionally substituted with C₁-C₆ alkyl;
Wherein R₉ and R₁₀ are independently selected from hydrogen and C₁-C₆ alkyl; or R₉, R₁₀ and the nitrogen atom bonded to them form a 4 to 6-membered heterocyclic ring containing optionally an additional heteroatom selected from N, O and S; and
X is NH or O, linked to the phenyl group at a para- or meta-position.

The second aspect of the present invention provides a method for treating or preventing diseases where vimentin is implicated, including methods for treating or preventing diseases related to cell endocytosis, exocytosis and endosomal trafficking, and for treating or preventing diseases related to an inadequate quantity and/or function of regulatory T cells. The said method comprises administering to a subject or individual in need for treatment or prevention an effective amount of the s-triazine derivatives represented by Formula A herein, or pharmaceutically acceptable carriers, prodrugs, enantiomers, diastereoisomers, tautomers or solvates thereof, or a composition containing a therapeutically or prophylactically effective amount of the s-triazine derivatives shown in Formula A herein, or a pharmaceutically acceptable pharmaceutical composition of carriers, prodrugs, enantiomers, diastereomers, tautomers or solvates.

The third aspect of the present invention provides the s-triazine derivatives shown in Formula A herein, or pharmaceutically acceptable carriers, prodrugs, enantiomers, diastereoisomers, tautomers solvate, or containing a therapeutically or prophylactically effective amount of the s-triazine derivatives represented by formula A herein, or pharmaceutically acceptable carriers, prodrugs, enantiomers, diastereoisomers, pharmaceutical compositions of tautomers or solvates, for treating or preventing diseases related to endocytosis, exocytosis and endosomal trafficking, and for treating or preventing diseases related to an inadequate quantity and/or function of regulatory T cells.

The compound of Formula A used in the method and use of any of the above aspects herein is preferably the compound described in any embodiment below, especially including the compounds described in formula I-1, I-2, I-3 and A-1 and each specific compound listed in the table.

The diseases related to endocytosis, exocytosis and endosomal trafficking described in any of the above aspects herein are diseases where vimentin is implicated, including cancer, pathogen infection, and other diseases caused by abnormality of one or more cellular processes. Preferably, the cancer has the following characteristics: its cancer cells use vimentin to achieve invasive growth, use endocytosis to uptake nutrients, use exocytosis to release exosomes as a medium to communicate with other cells, and create microenvironment suitable for the growth and metastasis of cancer cells; preferably, the said cancer includes: colon cancer, pancreatic cancer, ovarian cancer, gastric cancer, breast cancer, thyroid cancer, liver cancer, kidney cancer, lung cancer, prostate cancer, sarcoma, glioma, leukemia and multiple myeloma.

The pathogen described in any one of the above aspects herein is a bacterium and/or a virus, and the said pathogen enters the cell through endocytosis, traverses through endosomal pathway and/or releases progeny from the cell through exosomal pathway. Preferably, the said pathogen is selected from one or more of coronavirus (including SARS-CoV-2), HIV, influenza virus, hepatitis B virus, hepatitis C virus, human papillomavirus, Ebola virus, dengue virus, Escherichia coli, Salmonella enteritidis, Anaplasma phagocytophilum, Chlamydia trachomatis, Streptococcus pyogenes, Mycobacterium tuberculosis, Mycobacterium avium and Propionibacterium acnes; preferably, the said pathogen infection is the infection caused by one or more pathogens; preferably, the disease caused by the pathogen infection is an infectious disease or communicable disease, including but not limited to COVID-19, AIDS, hepatitis B, influenza, adhesion invasion coli (AIEC) infection.

The diseases associated with inadequate number and/or function of regulatory T cells as described in any of the above aspects herein are diseases where vimentin is implicated.

In some embodiments, the diseases associated with inadequate number and/or function of regulatory T cells are autoimmune diseases and inflammatory diseases, preferably including: inflammatory bowel disease (IBD), multiple sclerosis (MS), SARS-CoV infection (e.g. COVID-19), systemic lupus erythematosus (SLE), type 1 diabetes (T1D), psoriasis, graft-versus-host disease (GvHD), myasthenia gravis (MG), arthritis, scleroderma disease, dermatomyositis, vasculitis, neuritis, autoimmune hemolytic anemia, pernicious anemia with chronic atrophic gastritis, pulmonary hemorrhage nephritic syndrome, primary biliary cirrhosis, thyroid autoimmune disease, pemphigus, Sjorgen's syndrome, uveitis, allergic conjunctivitis, celiac disease, nonspecific colitis, fibrosis, autoimmune encephalomyelitis, atherosclerosis, chronic kidney disease, osteoporosis, allergies, fibromyalgia and neurodegeneration.

In some embodiments, the disease associated with inadequate number and/or function of regulatory T cells is a disease caused by cytokine storm, including acute respiratory distress syndrome and organ failure.

In some embodiments, the diseases associated with inadequate number and/or function of regulatory T cells are diseases with inflammatory components, including injury after cancer chemotherapy, infectious diseases and Alzheimer's disease.

### BRIEF DESCRIPTION OF THE FIGURES

FIG. 1: Binding of s-triazine derivatives to vimentin, such as C50, alters the distribution and mobility of vimentin in cells. A: The left panel shows the reorganization of the vimentin intermediate filament network. The results show that in U87 cells treated with compound C50, the vimentin network retracts from the periphery of the cell near the cell membrane to the center near the nucleus and connects with vesicle-like structures; the right panel shows no change in the GFAP network. B: The fluidity of vimentin filaments is impaired; in U87 cells expressing GFP-vimentin, the loss of fluorescence after photo bleach is rapidly restored in DMSO-treated cells but remains unfixed for a long time in C50-treated cells. C: Quantitative analysis of fluorescence recovery in DMSO- and C50-treated cells, measuring the fluorescence intensity of the target area at intervals of 30 s before and after photo bleach. Statistically significant *p<0.05, **p<0.01, ***p<0.001.

FIG. 2: Compound C52 inhibits cellular endocytosis and endosomal trafficking. GFP fluorescence intensity of pMAX-GFP transfected cells is shown. The time points when cells were treated with compound C52: A, entire transfection process; B, only before transfection; C, only after transfection. D is the dose-response curve under three conditions (A, B and C). Concentration unit of compound C52: μM; n=6 for each data point; data are expressed as mean±SEM.

FIG. 3: Multiple s-triazine derivatives, such as C45, C52, C69A, C69B, and C52M, can effectively inhibit the release of exosomes from liver cancer cells. After Huh7-NC12 was treated with various compounds for 48 h, the cells were used to determine the survival rate (A), and the supernatant of culture media was used to determine the content of exosomes (B). Compounds GW4869, C45, C52, and C69A were not cytotoxic even at the highest concentration (10,000 nM), while compounds C69B and C52M were cytotoxic at high concentrations (3,160 and 10,000 nM). All compounds inhibited the release of exosomes from HCC cells to some extent. Data are expressed as mean±SD, n=3.

FIG. 4: Compound C52 inhibits the secretion of exosomes from lung cancer and pancreatic cancer cells. (A) Representative transmission electron micrographs (arrows) of exosomes purified from culture medium of lung cancer A549 and pancreatic cancer PANC-1 cells treated with DMSO or 5 μM C52. Scale bar: 100 nm. (B) A549 and PANC-1 cells were treated with DMSO or 1 μM C52, and exosomes were isolated from cell culture supernatants of the same number of cells by serial ultracentrifugation and subjected to nanoparticle tracking analysis (NTA). (C) NTA quantification of three independent experiments. (D) Western blot analysis of A549 cells treated with DMSO or 0.1 μM, 1 μM and 10 μM C52 respectively for 48 h. Extracts from cells (intracellular) and culture media (extracellular) were blotted to detect the exosomal marker proteins CD9, CD63 and TSG101. (E) Quantification of exosomal marker proteins extracted from A549 cells and extracellular fluid in three independent experiments. (F) Western blot analysis of PANC-1 cells treated with DMSO or 0.1 μM, 1 μM, and 10 μM of C52 respectively for 48 h. Extracts from cells and extracellular fluid were blotted to detect exosomal marker proteins CD9, CD63 and TSG101. (G) Quantification of exosomal marker proteins extracted from PANC-1 cells and extracellular fluid in three independent experiments. Data are presented as mean±SEM (n=3). *P<0.05, **P<0.01 and ***P<0.001 (two-tailed Student's t-test).

FIG. 5: Vimentin regulates exosome formation and release, whereas compound C52, which binds to vimentin, only inhibits exosome release. (A) Representative western blot analysis of lysates from U87 Vim+/+ and U87 Vim+/− cells showing vimentin tetramers and vimentin monomers. (B) Quantification of protein levels of vimentin from (A) in three independent experiments. (C) Representative western blot analysis of cell lysates from equal numbers of U87 Vim+/+ and U87 Vim+/− cells treated with DMSO or 2 μM C52 for 72 h. Exosomal marker proteins CD9 and CD63 in cell lysates (intracellular) and β-actin were blotted as loading controls. (D-E) Protein levels of the exosomal marker proteins CD9 and CD63 in (C) were quantified in three independent experiments. (F) Extracellular vesicle lysates (Exo) were extracted from culture medium after treating equal amounts of U87 Vim+/+ and U87 Vim+/− cells with DMSO or 2 μM C52 for 72 h and blotted for their exosomal marker proteins CD9 and CD63. Shown is a representative western blot analysis. (G-H) Quantification of exosomal marker proteins CD9 and CD63 in (F) in three independent experiments. Data are presented as mean±SEM (n=3). *P<0.05, **P<0.01 and ***P<0.001 (two-tailed Student's t-test).

FIG. 6: Compound C52 binding to vimentin significantly inhibits the migration and invasion of cancer cells and can reduce the content of exosomes in blood in vivo. (A, B) Wound healing assay showing the migration (Left) and the quantification of cell confluency (right) of A549 and PANC-1 cells before and after treating A549 cells with DMSO or different concentrations of C52 for 48 h (A) or PANC-1 cells for 24 h (B). Scale bar: 300 μm. (C, D) Transwell invasive growth capacity assay showing the migration of (C) A549 or (D) PANC-1 cells treated with DMSO or different concentrations of C52 for 24 h (left) and quantification of migrated cells (right). (E) C52 (100 mg/kg, n=3) or vehicle (n=3) was administered to mice once a day for 14 days, and the changes of exosome content in plasma of mice were determined. Extracellular vesicles in plasma were purified by sucrose density gradient centrifugation, and the lysate was analyzed by Western blot for exosomal markers CD63 and TSG101 (left), and quantification of exosomal proteins in three independent Western blots (right). Scale bar: 300 μm. Data are presented as mean±SEM (n=3). *P<0.05, **P<0.01 (two-tailed Student's t-test).

FIG. 7: Compound C52 strongly inhibits the infection of cells by the SARS-CoV2 pseudovirus in vitro. (A) Fluorescence intensity of cells infected by pseudovirus-2019-nCoV. Compound C52 was present throughout the infection, and NH4Cl-treated cells were used as controls. (B-D) cell luciferase assay at 48 h after infection with pseudovirus-2019-nCoV. 2E+6, 2E+5, 2E+4 in the figure represent the virus titer used in the experiment. The concentration unit of compound C52 shown is in μM; n=3 for each data point; data are expressed as mean±SEM.

FIG. 8: In vivo, compound C52 can significantly improve clinical symptoms and pulmonary hemorrhage in aged mice infected with SARS-CoV2 virus. A: prophylactic administration, animal body weight change; B: prophylactic administration, animal pulmonary hemorrhage score; C: therapeutic administration, animal body weight change; D: therapeutic administration, animal pulmonary hemorrhage score. Statistically significant *P<0.05, **P<0.01, ***P<0.001 (compared to vehicle group of infected animals).

FIG. 9: Analytical identification of compound C52M. (A) Mass spectrum; (B) NMR spectrum.

FIG. 10: Vimentin-binding s-triazine derivatives have no cytotoxicity. C50 and C52 showed no toxic effects on (A) primary human hepatocytes and (B) primary human umbilical vein endothelial cells (HUVEC). Fluorescence units in each well were normalized to units of control hepatocytes or control HUVECs as relative cell viability. Data are expressed as mean SD.

FIG. 11: Vimentin-binding s-triazine derivatives (such as C52) have no effect on the growth of various types of cancer cells and major signaling pathways involved in cancer cell growth in vitro. (A): C52 does not inhibit the proliferation of cancer cells. For each concentration, from left to right are A549, AGS, U87, SMMC-7221, HUH7 and PANC-1. (B) and (D): Semi-quantification of oncoproteins in A549 cells (B) or PANC-1 cells (D) by Human XL Oncology. Cell lysates were prepared from A549, or PANC-1 cells treated with DMSO or 5 μM C52 for 48 h. (C) and (E): Panels summarize the relative signal intensities of specific proteins in A549 or PANC-1 cells, respectively, where, for each protein, the left column is the result of DMSO treatment, and the right column is the result of C52 treatment.

FIG. 12: Vimentin-binding s-triazine derivatives (e.g., C52) activate Treg cells in vivo and promote Treg cell regeneration in a syngeneic mouse tumor (CT26) model. (A) and (B): Tumor-bearing mice were orally treated daily with vehicle (blue line) or C52 (100 mg/kg/day, red line) during the study. On day 13 (arrows), all mice were each given a single intraperitoneal injection of saline (A) or low-dose cyclophosphamide (LDCP, B). Data are expressed as mean values of tumor volumes of animals in each group. A single dose of LDCP counteracted the effect of C52 for approximately 8 days (days 14 to 22, shaded segment). (C): C52 increased the regeneration of Treg cells in vivo. Both groups of mice from (B) were sacrificed on day 29, and CD3+ cells from lymph nodes were analyzed by FACS for CD4, CD25 and FoxP3. Data are presented as Ave±SD. Two-sided unpaired Student's t-test.

FIG. 13: Vimentin-binding s-triazine derivatives, such as C52, enhance Treg cells in vivo and reduce symptoms and tissue damage in mice with DSS-induced colitis. (A): The weight change of the animals during the experiment; (B): The proportion of Treg cells in mesenteric lymph node tumors of the animals to CD4+ T cells at the end of the experiment (D15); (C): The intestinal bleeding score of the animals during the experiment; (D): The disease activity index of the animals during the experiment; (E): Colon length picture (left), colon length statistics (right); (F): intestinal histopathological picture (left), intestinal histopathological score (right). Statistically significant *p<0.05, **p<0.01, ***p<0.001.

FIG. 14: Vimentin-conjugated s-triazine derivatives (e.g., C52) reduce animal weight loss and clinical symptoms in MOG-induced experimental autoimmune encephalomyelitis (EAE) mice. C57BL/6 mice (8 mice/group×5 groups) that induced EAE were treated daily with vehicle (G2), positive drug FTY720 (G3, 1 mg/kg) or compound C52 (G4˜G6, 10, 30 or 100 mg/kg) for 28 days. Data are presented as Ave±SD. Two-sided unpaired Student's t-test. G2-G6 vs G1 (normal): #P<0.05, ##P<0.01, ###P<0.001; G3-G6 vs G2: *P<0.05, **P<0.01, ***P<0.001. FTY720 and C52 at doses of 10 or 30 mg/kg reduced animal weight loss (A) and improved clinical disease scores (B).

### DETAILED DESCRIPTION OF THE INVENTION

It should be understood that within the scope of the present invention, the above-mentioned technical features of the present invention and the technical features specifically described in the following (such as embodiments) can be combined with each other to form a preferred technical solution.

The present invention finds that a series of s-triazine derivatives can change the spatial distribution and fluidity of vimentin filaments, thereby inhibiting cell endocytosis, endosomal trafficking and exosomal release. Specifically, the present invention uses representative s-triazine derivatives to demonstrate that such compounds affect the intracellular spatial distribution and physical properties of vimentin, inhibit endocytosis, interfere with endosomal trafficking, and block exosome secretion pathways. Using CRISPR gene editing, we found that vimentin can regulate the production and release of exosomes, and the s-triazine derivatives herein can reduce exosome release by targeting vimentin. This type of compound, in cancer, can effectively inhibit the invasive growth of various cancer cells and inhibit the release of cancer exosomes; in pathogen infection, it can effectively reduce the infection of cells by lentiviral (HIV)-based virus, and strongly inhibit the infection mediated by SARS-CoV-2 viral spike protein and human ACE2 receptor, reduce the exosome content in blood circulation, significantly improve the symptoms of SARS-CoV-2 infected mice and reduce their lung damage. Therefore, such s-triazine derivatives have therapeutic effects on various diseases including pathogen infection and cancer.

In addition, the present invention also found that a series of s-triazine derivatives can change the spatial distribution and physical properties of vimentin filaments and reduce the fluidity of vimentin without affecting cell growth and the main signaling pathways related to cell growth, can induce the activation and regeneration of Treg cells in vivo, and has significant therapeutic effects on animal models of various diseases. Therefore, such s-triazine derivatives can be used to treat or prevent various diseases related to the inadequate quantity and/or function of regulatory T cells, including autoimmune diseases and inflammatory diseases.

The s-triazine derivatives of the present invention preferably have a structural formula shown in the following formula A:

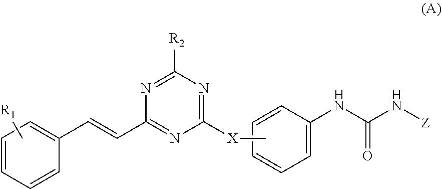

in the formula A:
R₁ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl or aminomethyl;
R₂ is —NR₄R₅, wherein R₄ and R₅ are independently selected from a group consisting of hydrogen, C₁-C₆ alkyl, and C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, hydroxyl, C₁-C₆ alkyl, or C₁-C₆ haloalkyl;
Z is aryl or heteroaryl optionally substituted by 1 to 3 R₃;
R₃ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl, aminomethyl or —CORₐ;
Wherein Rₐ is OH or NR₇R₈, wherein R₇ and R₈ are independently selected from hydrogen, C₁-C₆ alkyl optionally substituted with one or more substituents selected from halogen or NR₉R₁₀, and C₁-C₆ alkyl substituted with 3-(C₂-C₆ alkynyl)-3H-diaziridinyl; or R₇, R₈ and the nitrogen atom bonded to them form a 4- to 6-membered heterocyclic ring containing optionally an additional heteroatom selected from N, O and S, and optionally substituted with C₁-C₆ alkyl;
Wherein R₉ and R₁₀ are independently selected from hydrogen and C₁-C₆ alkyl; or R₉, R₁₀ and the nitrogen atom bonded to them form a 4- to 6-membered heterocyclic ring containing optionally an additional heteroatom selected from N, O and S; and
X is NH or O, linked to the phenyl group at a para- or meta-position.

Preferably, in Z of formula A, the aryl group is a 6- to 14-membered aryl group, such as phenyl or naphthyl; the heteroaryl group is a 5-10 membered heteroaryl group, preferably a nitrogen-containing heteroaryl group, including but not limited to imidazolyl, pyrazolyl, pyridyl, pyrimidinyl, pyrazinyl, pyridazinyl, pyrrolyl, triazolyl and tetrazolyl. Preferred Z is phenyl or pyridyl optionally substituted by 1 or 2 R₃.

The s-triazine derivatives of the present invention are preferably s-triazine derivatives described in U.S. Ser. No. 16/300,162, the entire contents of which are incorporated herein by reference. More specifically, the s-triazine derivatives of the present invention are 2,4,6-trisubstituted s-triazine compounds, which have the structure shown in the following formula I.

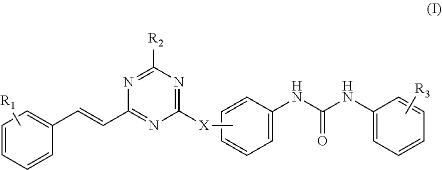

wherein:
R₁ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl or aminomethyl;
R₂ is —NR₄R₅, wherein R₄ and R₅ are independently selected from a group consisting of hydrogen, C₁-C₆ alkyl, and C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, hydroxyl, C₁-C₆ alkyl, or C₁-C₆ haloalkyl;
R₃ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl, aminomethyl or —CORₐ;
Wherein Rₐ is OH or NR₇R₈, wherein R₇ and R₈ are independently selected from hydrogen, C₁-C₆ alkyl optionally substituted with one or more substituents selected from halogen or NR₉R₁₀, and C₁-C₆ alkyl substituted with 3-(C₂-C₆ alkynyl)-3H-diaziridinyl; or R₇, R₈ and the nitrogen atom bonded to them form a 4- to 6-membered heterocyclic ring containing optionally an additional heteroatom selected from N, O and S, and optionally substituted with C₁-C₆ alkyl;
wherein R₉ and R₁₀ are independently selected from hydrogen and C₁-C₆ alkyl; or R₉, R₁₀ and the nitrogen atom bonded to them form a 4- to 6-membered heterocyclic ring containing optionally an additional heteroatom selected from N, O and S; and
X is NH or O, linked to the phenyl group at a para- or meta-position.

The s-triazine derivatives used in the present invention also include pharmaceutically acceptable salts, prodrugs, enantiomers, diastereoisomers, tautomers or solvates of compounds shown in formulas A and I.

In formula A and formula I, preferably, R₁ is hydrogen, halogen or nitro, more preferably H, F, Cl or nitro.

In formula A and formula I, preferably, R₂ is —NR₄R₅, wherein R₄ and R₅ are independently selected from hydrogen, C₁-C₆ alkyl, and C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, hydroxyl or C₁-C₆ alkyl. Preferably, R₄, R₅ and the nitrogen atom bonded to them form a saturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl; wherein R₆ is hydrogen or C₁-C₆ alkyl. Preferably, R₄ and R₅ are independently selected from hydrogen, C₁-C₆ alkyl; or R₄, R₅ and the nitrogen atom bonded to them form a saturated 4- to 6-membered heterocyclic ring containing optionally an additional heteroatom selected from NR₆, and O, wherein the heterocyclic ring may be substituted with hydroxyl, or C₁-C₆ alkyl; wherein R₆ is hydrogen or C₁-C₆ alkyl. The number of substituents on the heterocycle is usually 1, 2 or 3. Preferably, the 4-6 membered saturated heterocyclic ring includes but not limited to morpholinyl, pyrrolidinyl, piperazinyl, piperidinyl and azetidinyl.

In formula A and formula I, preferably, R₃ is hydrogen, halogen, nitro, amino, hydroxyl, C₁-C₆ alkyl, hydroxymethyl, aminomethyl or —CORₐ, wherein Rₐ is OH or NR₇R₈, wherein R₇ and R₈ are independently selected from hydrogen, C₁-C₆ alkyl optionally substituted with one or more substituents selected from halogen or NR₉R₁₀, and C₁-C₆ alkyl substituted with 3-(C₂-C₆alkynyl)-3H-diaziridinyl; or R₇, R₈ and the nitrogen atom bonded to them form a 4- to 6-membered saturated heterocyclic ring containing optionally an additional heteroatom selected from N and O, and optionally substituted with C₁-C₆ alkyl; R₉ and R₁₀ are independently selected from hydrogen and C₁-C₆ alkyl; or R₉, R₁₀ and the nitrogen atom bonded to them form a 4- to 6-membered heterocyclic ring containing optionally an additional heteroatom selected from N, O and S. Preferably, the heterocycles formed by R₇, R₈ and the nitrogen atom bonded to them and the heterocycles formed by R₉, R₁₀ and the nitrogen atom bonded to them include but are not limited to piperidinyl, piperazinyl, pyrrolidinyl and Morpholinyl. Preferably, when R₃ is a non-H substituent, it is usually located at the meta or para position of the phenyl group.

In formula A and formula I, preferably, X is NH, linked to the phenyl group at a para- or meta-position; or X is O, linked to the phenyl group at a para-position.

In a preferred embodiment, shown in formula A and formula I:
R₁ is hydrogen, halogen or nitro;
R₂ is —NR₄R₅, wherein R₄ and R₅ are independently selected from hydrogen, C₁-C₆ alkyl, C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl; wherein R₆ is hydrogen, hydroxyl or C₁-C₆ alkyl; and
R₃ is hydrogen, halogen, nitro, amino, hydroxyl, C₁-C₆ alkyl, hydroxymethyl, aminomethyl or —CORₐ; wherein Rₐ is OH or NR₇R₈, wherein R₇ and R₈ are independently selected from hydrogen, C₁-C₆ alkyl optionally substituted with one or more substituents selected from halogen or NR₉R₁₀, and C₁-C₆ alkyl substituted with 3-(C₂-C₆ alkynyl)-3H-diaziridinyl; or R₇, R₈ and the nitrogen atom bonded to them form a 4- to 6-membered heterocyclic ring containing optionally an additional heteroatom selected from N, O and S, and optionally substituted with C₁-C₆ alkyl; wherein R₉ and R₁₀ are independently selected from hydrogen and C₁-C₆ alkyl; or R₉, R₁₀ and the nitrogen atom bonded to them form a 4- to 6-membered heterocyclic ring containing optionally an additional heteroatom selected from N, O and S.

In a preferred embodiment, shown in formula A and formula I:
R₁ is hydrogen, halogen or nitro;
R₂ is —NR₄R₅, wherein R₄, R₅ and the nitrogen atom bonded to them form a saturated 4- to 6-membered heterocyclic ring containing optionally an additional heteroatom selected from NR₆ and O, wherein the heterocyclic ring is optionally substituted with a substituent selected from hydroxyl and C₁-C₆ alkyl; wherein R₆ is hydrogen or C₁-C₆ alkyl;
R₃ is halogen or —CORₐ; wherein Rₐ is OH or NR₇R₈, wherein R₇ and R₈ are independently selected from C₁-C₆ alkyl optionally substituted with NR₉R₁₀, and C₁-C₆ alkyl optionally substituted with 3-(C₂-C₆ alkynyl)-3H-diaziridinyl; or R₇, R₈ and the nitrogen atom bonded to them form a saturated 4- to 6-membered heterocyclic ring containing optionally an additional heteroatom selected from N or O, and optionally substituted with C₁-C₆ alkyl; wherein R₉ and R₁₀ are independently selected from hydrogen and C₁-C₆ alkyl; or R₉, R₁₀ and the nitrogen atom bonded to them form a saturated 4- to 6-membered heterocyclic ring containing optionally an additional heteroatom selected from N or O; and
X is NH, linked to the phenyl group at a para- or meta-position.

In some embodiments, preferably:
R₁ is hydrogen, halogen or nitro;
R₂ is —NR₄R₅, wherein R₄ and R₅ are independently selected from hydrogen, C₁-C₆ alkyl and C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl; wherein R₆ is hydrogen, hydroxyl or C₁-C₆ alkyl;
R₃ is hydrogen, halogen, nitro, amino, hydroxyl, C₁-C₆ alkyl, hydroxymethyl, aminomethyl or —CONR₇R₈, wherein R₇ and R₈ are independently selected from hydrogen and optionally substituted C₁-C₆ alkyl; or R₇, R₈ and the nitrogen atom bonded to them form a 4- to 6-membered heterocyclic ring containing optionally an additional heteroatom selected from N, O or S; wherein the C₁-C₆ alkyl may be optionally substituted with one or more halogen, C₁-C₆ alkylamino, di-C₁-C₆ alkylamino;
X is a para- or meta-NH or O.

In some embodiments, more preferably:
R₁ is hydrogen, halogen or nitro;
R₂ is —NR₄R₅, wherein R₄ and R₅ are independently selected from hydrogen and C₁-C₆ alkyl; or R₄, R₅ and the nitrogen atom bonded to them form a saturated 4- to 6-membered heterocyclic ring containing optionally an additional heteroatom selected NR₆, O and S, wherein the heterocyclic ring may be substituted with hydroxyl, halogen, nitro, amino or C₁-C₆ alkyl; wherein R₆ is hydrogen or C₁-C₆ alkyl;
R₃ is hydrogen, halogen, or —CONR₇R₈, wherein R₇ and R₈ are independently selected from hydrogen and optionally substituted C₁-C₆ alkyl; or R₇, R₈ and the nitrogen atom bonded to them form a saturated 4- to 6-membered heterocyclic ring containing optionally an additional heteroatom selected from N, O or S; wherein the C₁-C₆ alkyl may be optionally substituted with one or more C₁-C₆ alkylamino, di-C₁-C₆ alkylamino;
X is a para- or meta-NH or O.

In a preferred embodiment, the compound of formula I herein has the structure shown in the following formula I-1 or the following formula I-2:

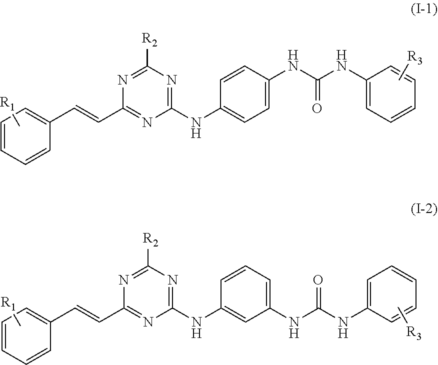

wherein:
R₁ is hydrogen, halogen or nitro;
R₂ is selected from morpholinyl, pyrrolidinyl, piperazinyl and azetidinyl optionally substituted by hydroxyl or C₁-C₆ alkyl; and
R₃ is halogen or CORₐ; wherein, Rₐ is OH or NR₇R₈, R₇ and R₈ are independently selected from C₁-C₆ alkyl optionally substituted by NR₉R₁₀ and C₁-C₆ alkyl substituted by 3-(C₂-C₆ alkynyl)-3H-diaziridinyl, or R₇, R₈ and the nitrogen atom bonded to them form a saturated 4- to 6-membered heterocyclic ring containing optionally an additional heteroatom selected from N or O; wherein R₉ and R₁₀ are independently selected from hydrogen and C₁-C₆ alkyl; or R₉, R₁₀ and the nitrogen atom bonded to them form a saturated 4- to 6-membered heterocyclic ring containing optionally an additional heteroatom selected from N or O.

Preferably, in the above formula I-2, R₃ is halogen.

Preferably, in the above formula I-1, R₁ is selected from H and halogen (preferably Cl); R₂ is selected from morpholino (preferably morpholino); R₃ is halogen or CORₐ, wherein, Rₐ is OH or NR₇R₈, wherein R₇ and R₈ and the nitrogen atom bonded to them form a saturated 4- to 6-membered heterocyclic ring containing optionally an additional heteroatom selected from N or O, and optionally substituted with C₁-C₆ alkyl, preferably piperidinyl, piperazinyl, pyrrolidinyl or azetidinyl, more preferably piperidinyl or piperazinyl substituted by C₁-C₄ alkyl.

In a preferred embodiment, the compound of formula I herein has the structure shown in the following formula I-3

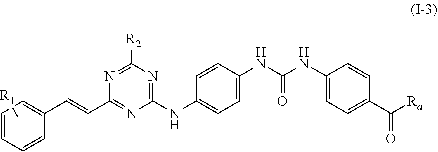

wherein:
R₁ is H;
R₂ is morpholinyl;
Rₐ is OH or NR₇R₈, wherein R₇ and R₈ are independently selected from C₁-C₆ alkyl optionally substituted by NR₉R₁₀ and by 3-(C₂-C₆ alkynyl)-3H-diaziridinyl, or R₇ and R₈ and the nitrogen atom bonded to them form a saturated 4- to 6-membered heterocyclic ring containing optionally an additional heteroatom selected from N or O and optionally substituted by C₁-C₆ alkyl; wherein R₉ and R₁₀ are independently selected from hydrogen and C₁-C₆ alkyl, or R₉, R₁₀ and the nitrogen atom bonded to them form a saturated 4- to 6-membered heterocyclic ring containing optionally an additional heteroatom selected from N or O.

In some embodiments of formula I-1, R₁ is selected from H, halogen and nitro; R₂ is morpholinyl; Rₐ is halogen or CORₐ; wherein, Rₐ is OH or NR₇R₈, wherein R₇ and R₈ are independently selected from C₁-C₆ alkyl optionally substituted by NR₉R₁₀ and by 3-(C₂-C₆ alkynyl)-3H-diaziridinyl, or R₇ and R₈ and the nitrogen atom bonded to them form a saturated 4- to 6-membered heterocyclic ring containing optionally an additional heteroatom selected from N or O. In certain embodiments, in these compounds, R₁ (when a non-hydrogen substituent) and R₃ are each independently located at the meta or para position of the phenyl group. In certain embodiments, in these compounds, when R₁ is a non-hydrogen substituent, it is located at the meta position of the phenyl group, and R₃ is located at the para position of the phenyl group. In certain embodiments, in these compounds, the saturated heterocycle includes, but is not limited to, piperazinyl, piperidinyl, pyrrolidinyl, and morpholinyl.

Preferably, the compound of formula A has the structure shown in the following formula A-1:

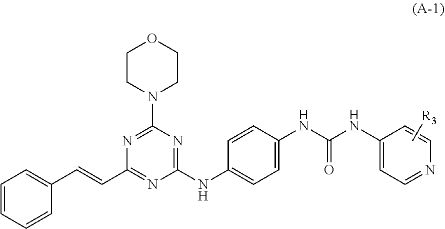

wherein:
R₃ is hydrogen, halogen, nitro, amino, hydroxyl, C₁-C₆ alkyl, hydroxymethyl, aminomethyl or —CONR₇R₈, wherein R₇ and R₈ are independently selected from hydrogen, C₁-C₆ optionally substituted alkyl, or R₇ and R₈ and the nitrogen atom bonded to them form a saturated 4- to 6-membered heterocyclic ring containing optionally an additional heteroatom selected from N, O and S; wherein the C₁-C₆ alkyl group can be optionally substituted by one or more halogens, C₁-C₆ alkylamino, di-C₁-C₆ alkylamino substitution.

Preferably, in formula A-1, R₃ is H or halogen.

Preferably, the compound of formula A of the present invention is selected from the following compounds L1-L42 and pharmaceutically acceptable salts, prodrugs, enantiomers, diastereomers, tautomers and solvates:

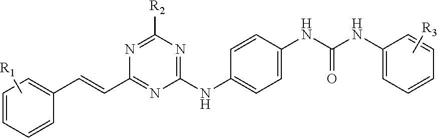

| ID(bioassay ID) | R₁ | R₂ | R₃ |
|---|---|---|---|
| L1(C50) | H | 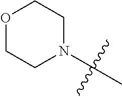 [SMILES: *N1CCOCC1] | 4-Cl |
| L2(C52) | H | 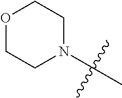 [SMILES: *N1CCOCC1] | 4-F |
| L3(C51) | H | 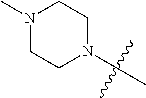 [SMILES: *N1CCN(C)CC1] | 4-Cl |
| L4(C53) | H | 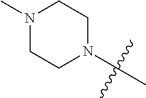 [SMILES: *N1CCN(C)CC1] | 4-F |
| L5(C57) | H | 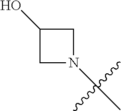 [SMILES: *N1CC(O)C1] | 4-Cl |
| L6(C54) | 3-Cl | 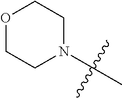 [SMILES: *N1CCOCC1] | 4-Cl |
| L7(C60) | 3-Cl | 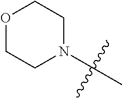 [SMILES: *N1CCOCC1] | 4-F |
| L8(C55) | 3-Cl | 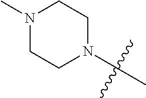 [SMILES: *N1CCN(C)CC1] | 4-Cl |
| L9(C61) | 3-Cl | 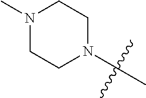 [SMILES: *N1CCN(C)CC1] | 4-F |
| L10(C43) | 4-Cl | 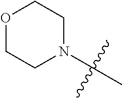 [SMILES: *N1CCOCC1] | 4-Cl |
| L11(C45) | 4-Cl | 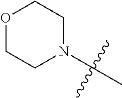 [SMILES: *N1CCOCC1] | 4-F |
| L12(C49) | 4-Cl | 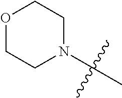 [SMILES: *N1CCOCC1] | 3-F |
| L13(C44) | 4-Cl | 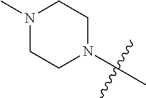 [SMILES: *N1CCN(C)CC1] | 4-Cl |
| L14(C42) | 4-Cl | 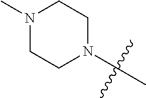 [SMILES: *N1CCN(C)CC1] | 4-F |
| L15(C48) | 4-Cl | 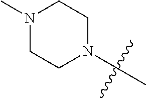 [SMILES: *N1CCN(C)CC1] | 3-F |
| L16(C40) | 3-NO₂ | 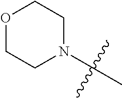 [SMILES: *N1CCOCC1] | 4-Cl |
| L17(C62) | 3-NO₂ | 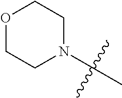 [SMILES: *N1CCOCC1] | 4-F |
| L18(C41) | 3-NO₂ | 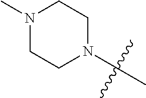 [SMILES: *N1CCN(C)CC1] | 4-Cl |
| L19(C63) | 3-NO₂ | 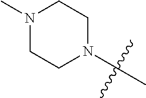 [SMILES: *N1CCN(C)CC1] | 4-F |
| L20(C38) | 3-NO₂ | 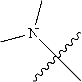 [SMILES: *N(C)C] | 4-Cl |
| L21(C39) | 3-NO₂ | 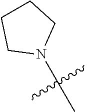 [SMILES: *N1CCCC1] | 4-Cl |
| L22(C58) | 3-NO₂ | 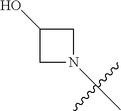 [SMILES: *N1CC(O)C1] | 4-Cl |

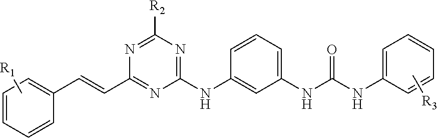

| ID(bioassay ID) | R₁ | R₂ | R₃ |
|---|---|---|---|
| L23(C64) | H | 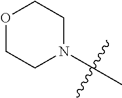 [SMILES: *N1CCOCC1] | 4-Cl |
| L24(C65) | H | 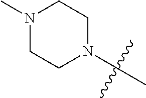 [SMILES: *N1CCN(C)CC1] | 4-Cl |
| L25(C59) | 3-Cl | 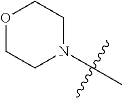 [SMILES: *N1CCOCC1] | 4-Cl |
| L26(C56) | 3-Cl | 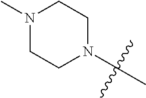 [SMILES: *N1CCN(C)CC1] | 4-Cl |
| L27(C66) | 3-NO₂ | 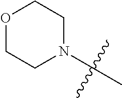 [SMILES: *N1CCOCC1] | 4-Cl |
| L28(C67) | 3-NO₂ | 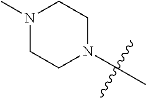 [SMILES: *N1CCN(C)CC1] | 4-Cl |

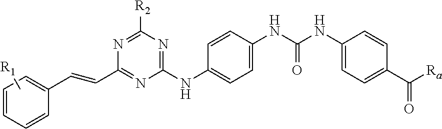

| ID | | | |
|---|---|---|---|
| (bioassay | | | |
| ID) | R₁ | R₂ | Rₐ |
| L29(C70) | H | 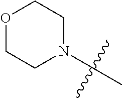 [SMILES: *N1CCOCC1] | 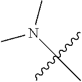 [SMILES: *N(C)C] |
| L30(C76) | H | 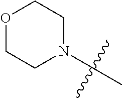 [SMILES: *N1CCOCC1] | 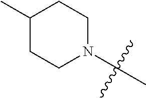 [SMILES: *N1CCC(C)CC1] |
| L31 (C69B) | H | 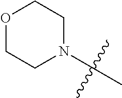 [SMILES: *N1CCOCC1] | 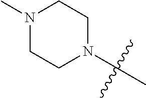 [SMILES: *N1CCN(C)CC1] |
| L32(C73) | H | 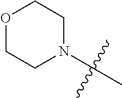 [SMILES: *N1CCOCC1] | 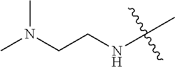 [SMILES: *NCCN(C)C] |
| L33(C74) | H | 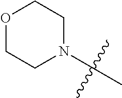 [SMILES: *N1CCOCC1] | 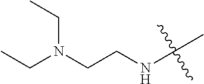 [SMILES: *NCCN(CC)CC] |
| L34(C77) | H | 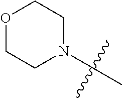 [SMILES: *N1CCOCC1] | 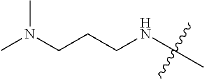 [SMILES: *NCCCN(C)C] |
| L35(C75) | H | 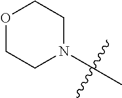 [SMILES: *N1CCOCC1] | 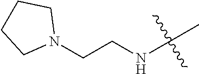 [SMILES: *NCCN1CCCC1] |
| L36(C72) | H | 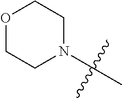 [SMILES: *N1CCOCC1] | 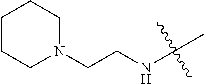 [SMILES: *NCCN1CCCCC1] |
| L37(C71) | H | 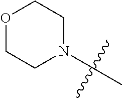 [SMILES: *N1CCOCC1] | 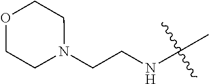 [SMILES: *NCCN1CCOCC1] |
| L38 (C69A) | H | 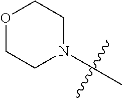 [SMILES: *N1CCOCC1] | OH |

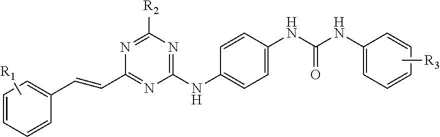

| ID(bioassay ID) | R₁ | R₂ | R₃ |
|---|---|---|---|
| L39(C84) | H | 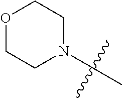 [SMILES: *N1CCOCC1] | 4-OCH₃ |
| L40(C85) | H | 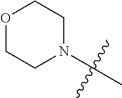 [SMILES: *N1CCOCC1] | 4-Cl |
| L41(C86) | H | 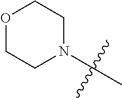 [SMILES: *N1CCOCC1] | 4-F |

And the compound L42 (C52M):

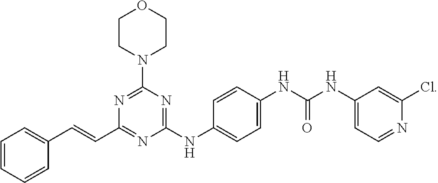 [SMILES: O=C(Nc1ccc(Nc2nc(/C=C/c3ccccc3)nc(N3CCOCC3)n2)cc1)Nc1ccnc(Cl)c1]

The compound of formula A described herein can be prepared according to the method disclosed in U.S. Ser. No. 16/300,162.

Herein, “alkyl” refers to C₁-C₁₂ alkyl, such as C₁-C₆ alkyl, such as methyl, ethyl, n-propyl, isopropyl, n-butyl, isobutyl, tert-butyl, n-pentyl, etc.

“Heterocycle” refers to a 4- to 6-membered heterocycle optionally containing heteroatoms selected from N, O and S. A heterocycle can be a saturated heterocycle or an unsaturated heterocycle. Exemplary heterocycles include, but are not limited to, e.g., morpholinyl, pyrrolidinyl, piperazinyl, piperidinyl, azetidinyl, pyrazolyl, and the like.

“Halogen” includes F, Cl, Br and I.

“Carboxyl” refers to —COOH.

In “3-(C₂-C₆ alkynyl)-3H-diaziridinyl”, the alkynyl position of the C₂-C₆ alkynyl is usually at the 1-position. In certain embodiments, the “3-(C₂-C₆ alkynyl)-3H-diaziridinyl” is “3-(1-butyn-4-yl)-3H-diaziridinyl-3-yl”.

Herein, NR₇R₈ and NR₉R₁₀ may be mono-C₁-C₆ alkylamino or di-C₁-C₆ alkylamino, said C₁-C₆ alkyl may be optionally substituted, for example by one or more halogens, mono-C₁-C₆ alkylamino or di-C₁-C₆ alkylamino substituted, or substituted by a 4- to 6-membered saturated heterocyclic ring containing N and optionally additional N or O. These heterocycles include, but are not limited to, piperidinyl, piperazinyl, pyrrolidinyl, morpholinyl, and the like. The heterocycle may also be optionally substituted, for example by C₁-C₆ alkyl.

Herein, “aryl” refers to a conjugated hydrocarbon ring system group having 6 to 18 carbon atoms, preferably 6 to 14 carbon atoms, more preferably 6 to 10 carbon atoms, for example 6, 7, 8, 9 or 10 carbon atoms. Aryl may be a monocyclic, bicyclic, tricyclic or multicyclic ring system and may also be fused to a cycloalkyl or heterocyclyl as defined above, provided that the aryl is single-bonded with the rest of the molecule. Examples of aryl groups described in various embodiments herein include, but are not limited to, phenyl, naphthyl, anthracenyl, phenanthrenyl, fluorenyl, 2,3-dihydro-1H-isoindolyl, 2-benzoxazolinone, 2H-1,4-benzoxazin-3(4H)-one-7-yl, etc.

In this application, the term “heteroaryl”, as a group or part of another group, means 5- to 16-membered conjugated ring groups having 1 to 15 carbon atoms (preferably 1 to 10 carbon atoms, e.g., 1, 2, 3, 4, 5, 6, 7, 8, 9 or 10 carbon atoms) and 1 to 6 heteroatoms selected from nitrogen, oxygen and sulfur. Unless specifically stated otherwise in this specification, heteroaryl may be a monocyclic, bicyclic, tricyclic or multicyclic ring system, and may be fused to a cycloalkyl or heterocyclyl as defined above, provided that heteroaryl group is connected to the rest of the molecule by a single bond through an atom on the aromatic ring. A nitrogen, carbon or sulfur atom in a heteroaryl can be optionally oxidized; the nitrogen atom can be optionally quaternized. For the purposes of the present invention, heteroaryl is preferably a stable 5- to 12-membered aromatic group containing 1 to 5 heteroatoms selected from nitrogen, oxygen and sulfur, more preferably 1 to 4 heteroatoms selected from a stable 5- to 10-membered aromatic group of heteroatoms selected from nitrogen, oxygen and sulfur or a 5- to 6-membered aromatic group containing 1 to 3 heteroatoms selected from nitrogen, oxygen and sulfur. Examples of heteroaryl groups described in various embodiments herein include, but are not limited to, thienyl, imidazolyl, pyrazolyl, thiazolyl, isothiazolyl, oxazolyl, oxadiazolyl, isoxazolyl, pyridyl, pyrimidinyl, pyrazinyl, pyridazinyl, benzimidazolyl, benzopyrazolyl, benzindolyl, benzomorpholinyl, benzisodiazolyl, indolyl, furyl, pyrrolyl, triazolyl, tetrazolyl, triazinyl, indolazinyl, isoindolyl, indazolyl, isindazolyl, purinyl, quinolinyl, isoquinolyl, naphthyl, naphthyridine base, quinoxaline base, pteridinyl, carbazolyl, carbolinyl, phenanthridinyl, phenanthrolinyl, acridinyl, phenazinyl, isothiazolyl, benzothiazolyl, benzothienyl, oxatriazolyl, cinnolinyl, quinazolinyl, phenylthio, indolizine, o-phenanthrenyl, isoxazolyl, phenoxazinyl, phenothiazinyl, 4,5,6,7-Tetrahydrobenzo[b]thienyl, naphthopyridyl, [1,2,4]triazolo[4,3-b]pyridazine, [1,2,4]triazolo[4,3-a]pyrazine, [1,2,4]triazolo[4,3-c]pyrimidine, [1,2,4]triazolo[4,3-a]pyridine, imidazo[1,2-a]pyridine, imidazo[1,2-b]pyridazine, imidazo[1,2-a]pyrazine, etc.

Herein, when a group is substituted, the number of substituents may vary, e.g., 1, 2, 3 or 4. Generally, unless otherwise stated, substituents may be selected from halogen, C₁-C₆ alkyl, hydroxyl, carboxyl, amino, mono-C₁-C₆ alkylamino, di-C₁-C₆ alkylamino, nitro, 3-(C₂-C₆ alkynyl)-3H-diaziridinyl, heterocyclic group (such as morpholinyl, pyrrolidinyl, piperazinyl, piperidinyl, azetidinyl, pyrazolyl, etc.) and C₆-C₁₄ aryl (such as phenyl), etc.

Related terms used herein such as “isomer”, “racemate”, “prodrug”, “solvate” are not significantly different from the general meanings of the terms in the art. Those of ordinary skill in the art should know the meaning of these terms. For example, the term “isomer” refers to one of two or more compounds that have the same molecular composition but differ in structure and properties. The term “racemate” refers to an equimolar mixture of an optically active chiral molecule and its enantiomer. The term “prodrug” is also called prodrug, drug precursor, bioprecursor, etc., and refers to a compound that has pharmacological effects after being transformed in vivo. The term “solvate” refers to a mixture of solvent and compound.

Herein, the term “pharmaceutically acceptable salt” includes pharmaceutically acceptable acid addition salts and pharmaceutically acceptable base addition salts.

Herein, “pharmaceutically acceptable acid addition salt” refers to a salt formed with an inorganic or organic acid that retains the biological effectiveness of the free base without other side effects. Inorganic acid salts include but not limited to hydrochloride, hydrobromide, sulfate, nitrate, phosphate, etc.; organic acid salts include but not limited to formate, acetate, 2,2-dichloroacetate, trifluoroacetate, propionate, caproate, caprylate, caprate, undecylenate, glycolate, gluconate, lactate, sebacate, hexanoate salt, glutarate, malonate, oxalate, maleate, succinate, fumarate, tartrate, citrate, palmitate, stearate, oleate, cinnamate, laurate, malate, glutamate, pyroglutamate, aspartate, benzoate, methanesulfonate, benzenesulfonate, p-toluenesulfonate, alginate, ascorbate, salicylate, 4-amino salicylate, naphthalene disulfonate, etc. These salts can be prepared by methods known in the field.

Herein, the term “pharmaceutically acceptable base addition salt” refers to salts formed with inorganic bases or organic bases that can retain the bioavailability of the free acid without causing any other side effects. Salts derived from inorganic bases include, but are not limited to, sodium salts, potassium salts, lithium salts, ammonium salts, calcium salts, magnesium salts, iron salts, zinc salts, copper salts, manganese salts, aluminum salts, etc. Preferred inorganic salts are ammonium salts, sodium salts, potassium salts, calcium salts, and magnesium salts. Salts derived from organic bases include, but are not limited to, primary, secondary, and tertiary amines, substituted amine classes, including naturally occurring substituted amine classes, cyclic amine classes, and alkaline ion exchange resins. Examples include ammonia, isopropylamine, trimethylamine, diethylamine, triethylamine, tripropylamine, ethanolamine, diethanolamine, triethanolamine, dimethylaminoethanol, 2-dimethylaminoethanol, 2-diethylaminoethanol, dicyclohexylamine, lysine, arginine, histidine, caffeine, procaine, choline, betaine, ethylenediamine, glucosamine, methylglucosamine, theobromine, purine, pyrimidine, pyridine, N-ethylpyridine, polyamine resins, etc. Preferred organic bases include isopropylamine, diethylamine, ethanolamine, trimethylamine, dicyclohexylamine, choline, and caffeine. These salts can be prepared using methods known in the field.

Embodiments of the prodrug of the present invention may include simple esters of compounds containing carboxylic acid (for example, esters obtained by condensation with C₁-C₄ alcohols according to known methods in the field); esters of compounds containing hydroxyl groups (for example, esters obtained by condensation with C₁-C₄ carboxylic acids, C₃-C₆ diacids, or their anhydrides such as succinic anhydride and fumaric anhydride according to known methods in the field); imines of compounds containing amino groups (for example, imines obtained by condensation with C₁-C₄ aldehydes or ketones according to known methods in the field); aminoformates of compounds containing amino groups, such as those described by Leu et al. (J. Med. Chem., 42:3623-3628; 1999) and Greenwald et al. (J. Med. Chem., 42:3657-3667; 1999); aldehyde-hydrosols or ketone-hydrosols of compounds containing alcohols (for example, hydrosols obtained by condensation with chloromethyl methyl ether or chloromethyl ethyl ether according to known methods in the field).

The present invention relates to a use of a compound of formula A described herein (including the compound of formula I, compound of formula I-1, compound of formula I-2, compound of formula I-3 and compound of formula A-1) or a pharmaceutically acceptable salt thereof, prodrugs, enantiomers, diastereoisomers, tautomers or solvates in the preparation of medicines for the treatment or prevention of vimentin-mediated diseases, including the diseases related to endocytosis, exocytosis and endosomal trafficking, as well as the diseases associated with inadequate number and/or function of regulatory T cells. The present invention also relates to a compound of formula A described herein (including the compound of formula I, compound of formula I-1, compound of formula I-2, compound of formula I-3 and compound of formula A-1) or pharmaceutically acceptable salts, prodrugs, enantiomers, diastereoisomers, tautomers or solvates thereof, and pharmaceutical compositions thereof in the treatment or prevention of vimentin-mediated diseases, including the treatment or prevention of diseases related to cell endocytosis, exocytosis and endosomal trafficking, and the treatment or prevention of diseases related to inadequate number and/or function of regulatory T cells. Also included herein are methods of treating or preventing diseases where vimentin is implicated, including methods of treating or preventing diseases associated with cellular endocytosis, exocytosis, and endosomal trafficking, as well as methods of treating or preventing diseases related to regulatory T cell numbers and/or function. The said method comprises administering to a subject in need thereof an effective amount of a compound of formula A described herein (including said compound of formula I, compound of formula I-1, compound of formula I-2, compound of formula I-3 compound and formula A-1 compound) or pharmaceutically acceptable salts, prodrugs, enantiomers, diastereomers, tautomers or solvates, or a pharmaceutical composition described herein.

Herein, “pharmaceutical composition” refers to a formulation of a compound of the present invention and an art-recognized vehicle for delivering a biologically active compound to a mammal (e.g., a human). Such vehicle includes all pharmaceutically acceptable carriers, diluents or excipients therefor.

As used herein, “therapeutically effective amount” means at the necessary dosage and for the necessary period of time, the amount to achieve the desired therapeutic effect (such as reduced tumor size, increased life span or life expectancy, reduced pulmonary bleeding, or improved clinical symptoms). A therapeutically effective amount of a compound can vary depending on factors such as the disease state, age, sex and weight of the subject, and the ability of the compound to elicit a desired response in the subject. Dosage regimens may be adjusted to provide the optimal therapeutic response. A therapeutically effective amount is also the one in which any toxic or detrimental effects of the compound are outweighed by the therapeutically beneficial effects. A “prophylactically effective amount” refers to an amount effective, at dosages and for periods of time necessary, to achieve the desired prophylactic result (e.g., smaller tumors, prolonged lifespan or life expectancy). Typically, a prophylactic dose is administered to a subject before or at an early stage of the disease, such that the prophylactically effective amount may be less than the therapeutically effective amount. In preferred embodiments, the amount of the compound described herein administered is sufficient to interfere with the growth and spread of cancer cells or to disrupt one or more cellular processes required to cause pathogenic infection, or to activate Treg cell function in vivo, promote Treg cell restoration, but not enough to cause potential adverse effects. As used herein, “potential adverse effects” means that the dose of the compound is high enough to affect other immune cells that may counter Treg activation.

As used herein, “treatment” encompasses the treatment of a disease or condition of interest in a mammal, preferably a human, suffering from the disease or condition of interest, and includes:
(i) preventing the occurrence of said disease or condition in a mammal, especially when said mammal is susceptible to, but not yet diagnosed with, said condition;
(ii) inhibiting said disease or condition, i.e., arresting its development;
(iii) alleviating the disease or condition, i.e., causing regression of the disease or condition; or
(iv) Alleviating symptoms caused by the disease or condition, i.e., alleviating suffering without addressing the underlying disease or condition.

As used herein, the terms “administering”, “administration” and the like refer to methods capable of delivering a compound or composition to the desired site of biological action.

Administration methods known in the art can be used in the present invention. These methods include, but are not limited to, oral route, transduodenal route, parenteral injection (including intrapulmonary, intranasal, intrathecal, intravenous, subcutaneous, intraperitoneal, intramuscular, intraarterial injection or infusion), topical and rectal administration.

Administration techniques useful for the compounds and methods described herein are well known to those skilled in the art, as described, for example, in Goodman and Gilman, The Pharmacological Basis of Therapeutics, current ed.; Pergamon; and Remington's, Pharmaceutical Sciences (current edition), Mack Publishing Co., Easton, Pa.

Herein, preferably, the diseases related to cell endocytosis, exocytosis and endosomal trafficking are diseases where vimentin is implicated. Specifically, the compound of the present invention can inhibit the endocytosis, endosomal trafficking and/or cancer exosomal release by targeting vimentin, so as to treat or prevent the diseases associated with these cellular processes.

Herein, preferably, the diseases related to cell endocytosis, exocytosis and endosomal trafficking include cancer, pathogenic infection, and other diseases in which abnormalities in one or more cellular processes lead to pathogenesis. Preferably, the cancer has the following characteristics: the cancer cells use vimentin to achieve invasive growth, use endocytosis to uptake nutrients, use exocytosis to release exosomes as a mediator to communicate with other cells, and create microenvironment suitable for the growth and metastasis of cancer cells. In some embodiments, the cancer includes colon cancer, pancreatic cancer, ovarian cancer, gastric cancer, breast cancer, thyroid cancer, liver cancer, kidney cancer, lung cancer (such as non-small cell lung cancer), prostate cancer, sarcoma, glioma, leukemia and multiple myeloma. Preferably, the cancer is a cancer where vimentin is implicated. In some embodiments, the cancer is liver cancer, lung cancer, glioma, and pancreatic cancer.

Herein, the pathogens may be bacteria and/or viruses. Typically, the said pathogen enters the cell through endocytosis, traverses within the cell through the endosomal pathway and/or releases progeny from the cell through the exosomal pathway. Preferably, the pathogen can be selected from one or multiple of: coronavirus (including SARS-CoV-2), HIV, influenza virus, hepatitis B virus, hepatitis C virus, human papillomavirus, Ebola virus, dengue virus, Escherichia coli, Salmonella enterica, Anaplasma phagocytophilum, Chlamydia trachomatis, Streptococcus pyogenes, Mycobacterium tuberculosis, Mycobacterium avium, and Propionibacterium acnes. Preferably, the infection is an infection caused by one or more of these pathogens. Preferably, the disease caused by the pathogen is an infectious disease or a communicable disease, including but not limited to COVID-19, AIDS, hepatitis B, influenza, and adherent invasive Escherichia coli (AIEC) infection. In some embodiments, the diseases associated with endocytosis, exocytosis and endosomal trafficking exhibit various symptoms and/or tissue damage caused by pathogen infection, such as respiratory symptoms and lung damages caused by coronaviruses, especially SARS-CoV-2.

Therefore, herein, the “cell” may be a cell of a normal tissue or a cell of a diseased tissue. The compounds described herein can prevent exosomes produced by cells in diseased tissue from entering the normal tissue cells and spreading among cells in normal tissue by inhibiting endocytosis and endosomal trafficking of normal tissue cells, thereby prevent, halt or slow down the progress of the disease, and can also inhibit the endocytosis and endosome trafficking of the cells in diseased tissue, thereby preventing or delaying the further deterioration of the disease. In another embodiment, the compounds described herein can prevent, halt or slow down the infection or synchronization of cells in normal tissues by inhibiting the excretion of exosomes from diseased or infected cells, and thereby prevent or delay the progression of the disease.

In a preferred embodiment, the present invention relates to a compound of formula A described herein (including said compound of formula I, compound of formula I-1, compound of formula I-2, compound of formula I-3 and compound of formula A-1) or its pharmaceutically acceptable salts, prodrugs, enantiomers, diastereoisomers, tautomers or solvates for its use in the preparation of medicines for treating or preventing cancer, and in the preparation of medicines for treating or preventing pathogen infection or a disease caused by pathogen infection. The present invention also relates to a compound of formula A described herein (including the compound of formula I, compound of formula I-1, compound of formula I-2, formula I-3 compound, and formula A-1 compound) or its pharmaceutically acceptable salts, prodrugs, enantiomers, diastereoisomers, tautomers or solvates, and pharmaceuticals thereof combination for treating or preventing cancer or pathogen infection or a disease caused by pathogen infection. Also included herein is a method for treating or preventing cancer or pathogenic infection or a disease caused by pathogenic infection, said method comprising administering to a subject in need a compound of formula A described herein (including said compound of formula I, formula I-1 compound, formula I-2 compound, formula I-3 compound and formula A-1 compound) or its pharmaceutically acceptable salts, prodrugs, enantiomers, diastereoisomers, tautomers or solvates, or administering a therapeutically or prophylactically effective amount of a pharmaceutical composition described herein.

Herein, preferably, the diseases associated with inadequate number and/or function of regulatory T cells refer to various diseases or conditions caused by inadequate number and/or function of Treg cells. In some embodiments, these diseases are diseases where vimentin is implicated. Specifically, vimentin restrains the function of Treg cells by retaining the suppressive molecules in Treg cells at the distal pole complex (DPC), so preventing them from being released to the immunological synapse formed between Treg cells and antigen-presenting cells (APC) and therefore preventing them from exerting their suppressive functions (McDonald-Hyman, C. et al., The Vimentin Intermediate Filament Network Restrains Regulatory T Cell Suppression of Graft-Versus-Host Disease. J. Clin. Invest. 128:4604-4621, 2018). The s-triazine derivatives described herein can bind to vimentin and change the spatial distribution and physical properties of vimentin in the cell, causing disassembly of the DPC and therefore release of these suppressive molecules, therefore activating Treg cells, so as to achieve the effect of treating or preventing the diseases related to the inadequate quantity and/or function of regulatory T cells.

Herein, preferably, the diseases associated with inadequate number and/or function of regulatory T cells may be autoimmune diseases and inflammatory diseases, including: inflammatory bowel disease (IBD), multiple sclerosis (MS), SARS-CoV infection (e.g. COVID-19), systemic lupus erythematosus (SLE), type 1 diabetes (T1D), psoriasis, graft-versus-host disease (GvHD), myasthenia gravis (MG), arthritis, scleroderma, dermatomyositis, vasculitis, neuritis, autoimmune hemolytic anemia, pernicious anemia with chronic atrophic gastritis, pulmonary hemorrhagic nephritic syndrome, primary biliary cirrhosis, thyroid autoimmune disease, pemphigus, Sjorgen syndrome, uveitis, allergic conjunctivitis, celiac disease, nonspecific colitis, fibrosis, autoimmune encephalomyelitis (EAE), atherosclerosis, chronic kidney disease, osteoporosis, allergies, Fibromyalgia and neurodegeneration. Preferably, the disease is IBD, specifically including Crohn's disease and ulcerative colitis. In some embodiments, the disease is multiple sclerosis (MS), SARS-CoV infection (e.g., COVID-19), systemic lupus erythematosus (SLE), type 1 diabetes (T1D), psoriasis, graft versus host disease (GvHD), myasthenia gravis (MG).

In addition to autoimmune and inflammatory diseases, Treg activation can also benefit other diseases caused by inflammatory mediators. Herein, inflammatory mediators may be molecules known in the art that participate in and mediate inflammatory reactions, including but not limited to various cytokines, platelet activating factors and leukocyte products, vasoactive amines, arachidonic acid metabolites, and the like. Such diseases caused by inflammatory mediators include damage after cancer chemotherapy, infectious disease and Alzheimer's disease.

In addition, an overreacting immune system is a major cause of tissue damage, organ failure and death in patients with certain diseases. In particularly preferred embodiments, the regimens provided herein help reduce cytokine storms, thereby avoiding, arresting, or slowing tissue damage, organ failure, and death in patients. More specifically, the regimens of the present invention are useful in the treatment of COVID-19 infection, especially in the treatment or prevention of tissue damage, organ failure and death in patients resulting from the infection.

Herein, the individual or subject is preferably a mammal, more preferably a human.

Herein, therapeutic benefit may be achieved by simultaneous or sequential administration of at least 1, 2, 3 or more compounds described herein. The compounds or pharmaceutical compositions described herein may also be combined with other therapies to provide a combined therapeutically effective dose. For example, the compounds or pharmaceutical compositions described herein may be administered in combination with other drugs, preferably antibacterial or viral drugs, or in combination with immunomodulators.

The pharmaceutical composition provided herein may contain the compound of formula A described in any embodiment herein (including the compound of formula I, compound of formula I-1, compound of formula I-2, compound of formula I-3 and compound of formula A-1) or a pharmaceutically acceptable salt, prodrug, enantiomer, diastereoisomer, tautomer or solvate thereof and a pharmaceutically acceptable carrier, diluent or excipient.

Herein, “pharmaceutically acceptable carrier, diluent or excipient” includes, but is not limited to, any adjuvant, carrier, excipient, glidant, sweetener, diluent, preservative, dye/colorant, flavor enhancer, surfactant, wetting agent, dispersing agent, suspending agent, stabilizer, isotonic agent, solvent, or emulsifying agent. Usually, a pharmaceutically acceptable carrier is an inert diluent.

In a preferred embodiment, the pharmaceutical composition herein comprises Compound C45, C50, C52, C52M, C69A and/or Compound C69B. In some preferred embodiments, the pharmaceutical composition of the present invention contains the compound shown in formula I-1, its pharmaceutically acceptable salt, prodrug, enantiomer, diastereoisomer, tautomer or solvate, wherein R₁ is selected from H and halogen (preferably Cl), more preferably H; R₂ is selected from morpholino group (preferably morpholino); R₃ is halogen or CORₐ, more preferably halogen; Rₐ is OH or NR₇R₈, wherein R₇, R₈ and the nitrogen atom bonded to them form a 4- to 6-membered saturated heterocyclic ring containing optionally an additional heteroatom selected from N or O, and optionally substituted with C₁-C₆ alkyl, preferably, piperidyl, piperazinyl, pyrrolidinyl or azetidinyl, more preferably, piperidinyl or piperazinyl substituted by C₁-C₄ alkyl. More preferably, the pharmaceutical composition of the present invention contains compounds C50 and/or C52.

The pharmaceutical compositions herein can take a variety of forms to suit the chosen route of administration. Those skilled in the art will recognize various synthetic methods that can be used to prepare non-toxic pharmaceutically acceptable compositions of the compounds described herein. Those skilled in the art will recognize that a variety of non-toxic pharmaceutically acceptable solvents can be employed to prepare solvates of the compounds of the present invention.

The pharmaceutical composition of the present invention may be in various suitable dosage forms, including pills, capsules, elixirs, syrups, lozenges and the like. The pharmaceutical composition of the present invention can be administered by various suitable routes, including oral, topical, parenteral, inhalation or spray or rectal administration and the like. The term “parenteral” as used herein includes subcutaneous injection, intradermal, intravascular (e.g. intravenous), intramuscular, spinal, intrathecal injection or similar injection or infusion techniques.

The pharmaceutical compositions containing the compounds of the present invention are preferably in a form suitable for oral use, such as tablets, lozenges, aqueous or oily suspensions, dispersible powders or granules, emulsions, hard or soft capsules or syrups or elixirs.

Compositions for oral administration may be prepared according to any method known in the art for the preparation of pharmaceutical compositions, and such compositions may contain one or more agents selected from the group consisting of sweeteners, flavoring agents, colorants, and preservatives, in order to provide pharmaceutically elegant and palatable preparations. Tablets may contain the active ingredient in admixture with non-toxic pharmaceutically acceptable excipients which are suitable for the manufacture of tablets. These excipients can be, for example, inert diluents, including calcium carbonate, sodium carbonate, lactose, calcium or sodium phosphate; granulating and disintegrating agents, such as cornstarch or alginic acid; binders, such as starch, gelatin or arabic acid; gums; lubricants such as magnesium stearate, stearic acid or talc. Tablets may be uncoated or coated by known techniques to delay disintegration and absorption in the gastrointestinal tract and thus provide sustained action over a longer period of time. For example, a time delay material such as glyceryl monostearate or glyceryl distearate may be employed.

Oral formulations may also be presented as hard gelatin capsules in which the active ingredient is mixed with an inert solid diluent such as calcium carbonate, calcium phosphate, or kaolin, or as soft gelatin capsules in which the active ingredient is mixed with water or oil medium such as peanut oil, liquid paraffin, or olive oil.

Aqueous suspensions contain the active materials in admixture with excipients suitable for the manufacture of aqueous suspensions. Such excipients are suspending agents such as sodium carboxymethylcellulose, methylcellulose, hydroxypropylmethylcellulose, sodium alginate, polyvinylpyrrolidone, tragacanth and acacia. Dispersants or wetting agents may be naturally occurring phospholipids, such as lecithin, or condensation products of alkylene oxides with fatty acids, such as polyoxyethylene stearate, or condensation products of ethylene oxide with long-chain aliphatic alcohols such as octaoctadecylethyleneoxycetyl alcohol, or condensation products of ethylene oxide with partial esters derived from fatty acids and hexitols, such as polyoxyethylene sorbitan monooleate, or condensation products of ethylene oxide with partial esters derived from fatty acids and hexitol anhydrides, such as polyethylene sorbitan monooleate. Aqueous suspensions may also contain one or more preservatives, such as ethyl or n-propyl p-hydroxybenzoate, one or more coloring agents, one or more flavoring agents and one or more sweeteners such as sucrose or saccharin.

Oily suspensions may be formulated by suspending the active ingredient in a vegetable oil such as arachis oil, olive oil, sesame oil or coconut oil, or mineral oil such as liquid paraffin. The oily suspensions may contain a thickening agent, such as beeswax, hard paraffin or cetyl alcohol. Sweetening and flavoring agents such as those mentioned above may be added to provide a palatable oral preparation. These compositions can be preserved by the addition of antioxidants such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Other excipients, for example sweetening, flavoring, and coloring agents, may also be present.

The pharmaceutical compositions of the invention may also be in the form of oil-in-water emulsions. The oily phase may be a vegetable oil, such as olive oil or arachis oil, or a mineral oil, such as liquid paraffin or mixtures of these. Suitable emulsifiers may be naturally occurring gums such as acacia or tragacanth; naturally occurring phospholipids such as soybean, lecithin and esters or partial esters derived from fatty acids and hexitols; acid anhydrides such as sorbitan mono-oil acid esters; condensation products of said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol, or sucrose. Such formulations may also contain a demulcent, a preservative and flavoring and coloring agents. The pharmaceutical compositions may be in the form of sterile injectable aqueous or oleaginous suspensions. This suspension may be formulated according to the known art using those suitable dispersing or wetting agents and suspending agents which have been mentioned above. The sterile injectable preparation may also be a sterile injectable solution or suspension in a nontoxic parenterally acceptable diluent or solvent, for example a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The pharmaceutical compositions of the invention may also be administered in the form of suppositories, for example for rectal administration. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Alternatively, the compositions can be administered parenterally in a sterile vehicle. Depending on the vehicle and concentration used, the drug can be suspended or dissolved in the vehicle. Advantageously, adjuvants such as local anesthetics, preservatives and buffering agents can be dissolved in the vehicle.

For administration to non-human animals, compositions containing therapeutic compounds can be added to the animal's feed or drinking water. Furthermore, it would be convenient to formulate animal feed and drinking water products so that the animals receive appropriate amounts of the compound in their diet. To further facilitate administration, the compounds may be present in the composition as a premix for addition to feed or drinking water. The composition may also be added as a food or beverage supplement for humans.

Dosage levels useful in the treatment of the above conditions include about 1 mg to about 500 mg per day, about 5 mg to about 150 mg per day, more preferably about 5 mg to about 100 mg per day. The amount of active ingredient which can be combined with a carrier material to produce a single dosage form will vary depending upon the condition being treated and the mode of administration. The dose for the treatment of autoimmune inflammatory diseases is preferably at least three times less than the dose for the treatment of proliferative diseases.

The frequency of dosing can also vary depending on the compound employed and the disease being treated. However, for the treatment of most diseases, a dosing regimen of 3 times per day or less is preferred. It should be understood that the specific dosage level for any particular patient will depend on a variety of factors, including the activity of the particular compound employed, age, body weight, general health, sex, diet, time of administration, route of administration and rate of excretion, drug combination and the severity of the particular condition being treated.

Preferred compounds of the invention will possess desirable pharmacological properties including, but not limited to, oral bioavailability, low toxicity, low serum protein binding and desirable in vitro and in vivo half-lives. For compounds used to treat diseases of the central nervous system, it is necessary to penetrate the blood-brain barrier, while for the treatment of peripheral diseases compounds with low levels of exposure in brain tissue are generally preferred. For the treatment of organ-specific diseases, compounds with enriched exposure in an organ and minimal exposure in other organs or systemically are preferred.

The amount of the composition required for treatment will vary not only with the compound chosen but also with the route of administration, the nature of the disease being treated and the age and condition of the patient and will ultimately depend on the attending physician.

Hereinafter, the present invention will be illustrated in the form of specific embodiments. It should be understood that these examples are illustrative only and are not intended to limit the scope of the present invention. The methods and materials used in the examples, unless otherwise stated, are conventional methods in the art and commercially available materials.

### PREPARATION EXAMPLE

The compound of formula A can be prepared according to the method disclosed in U.S. Ser. No. 16/300,162.

The preparation method of compound L42 (C52M) is exemplarily given below.

### Step 1

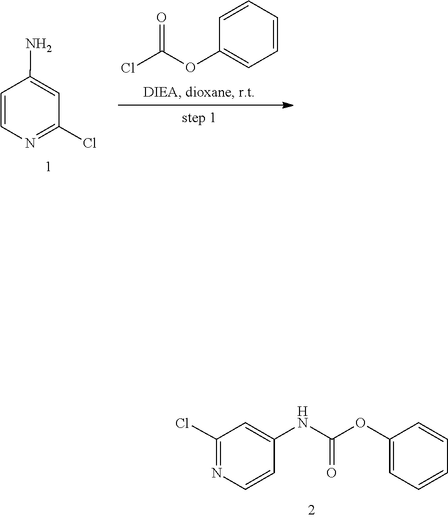 [SMILES: Nc1ccnc(Cl)c1 ; O=C(Cl)Oc1ccccc1 ; O=C(Nc1ccnc(Cl)c1)Oc1ccccc1]

A solution of 1 (3.0 g, 23.3 mmol) in dioxane (50 mL) was cooled to 10° C., then DIEA (4.0 g, 46.6 mmol) and phenyl chloroformate (4.0 g, 25.6 mmol) were sequentially added dropwise under nitrogen. After the addition, the mixture was warmed to room temperature and stirring was continued until complete. The mixture was cooled to 10° C. and quenched with saturated aqueous NaHCO3. The NaHCO3 solution (50 mL) was separated, and the aqueous layer was extracted with EA (100 mL*2). The combined organic layers were washed with brine, dried over sodium sulfate, concentrated, and the residue was triturated with MeOH (20 mL) to give the desired product, compound 2 (2.5 g, 43%), as an off-white solid.

### Step 2

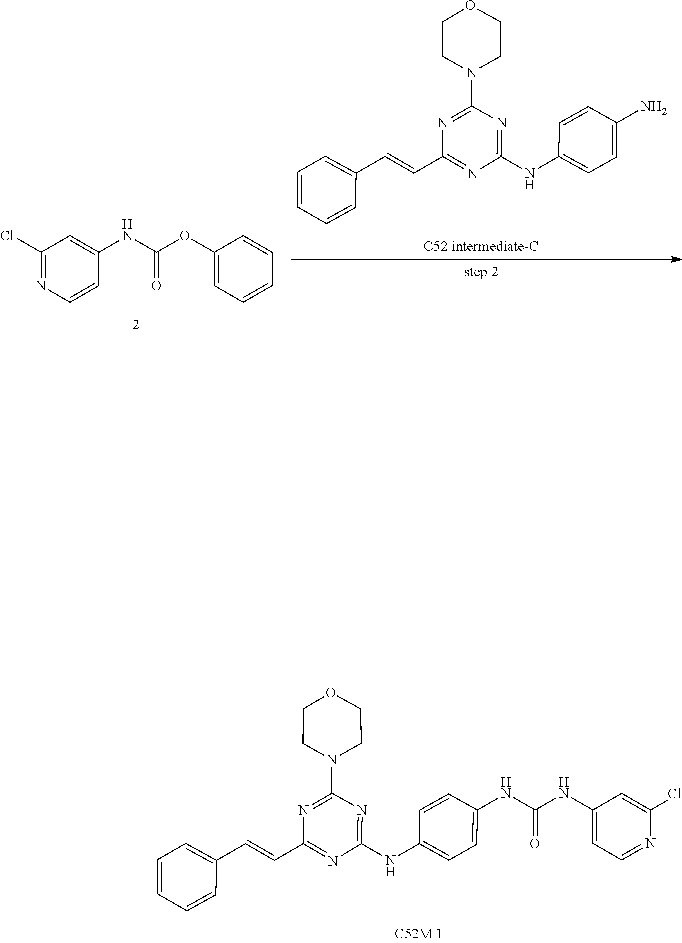 [SMILES: Nc1ccc(Nc2nc(/C=C/c3ccccc3)nc(N3CCOCC3)n2)cc1 ; O=C(Nc1ccnc(Cl)c1)Oc1ccccc1 ; O=C(Nc1ccc(Nc2nc(/C=C/c3ccccc3)nc(N3CCOCC3)n2)cc1)Nc1ccnc(Cl)c1]

To a solution of C52 Intermediate-C (prepared according to the method disclosed in U.S. Ser. No. 16/300,162, 1.0 g, 2.67 mmol) in DMSO (15 mL) under nitrogen, DIEA (861 mg, 6.68 mmol, 2.5 eq) was added compound 2 (1.3 g, 5.34 mmol, 2 eq). The mixture was stirred at 60° C. for 2 h. TLC and LCMS detection indicated that the reaction was complete. The mixture was diluted with water (40 mL) and the solid formed was filtered, washed with MeOH and dried to give the desired product C52M (900 mg, 64%), as an off-white solid.

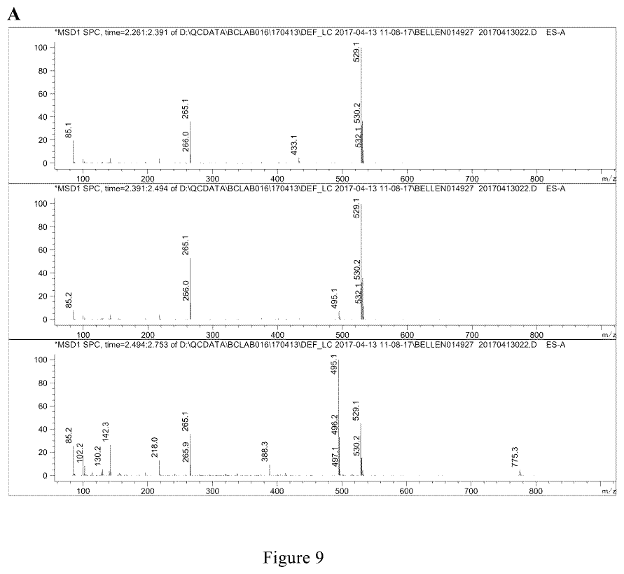

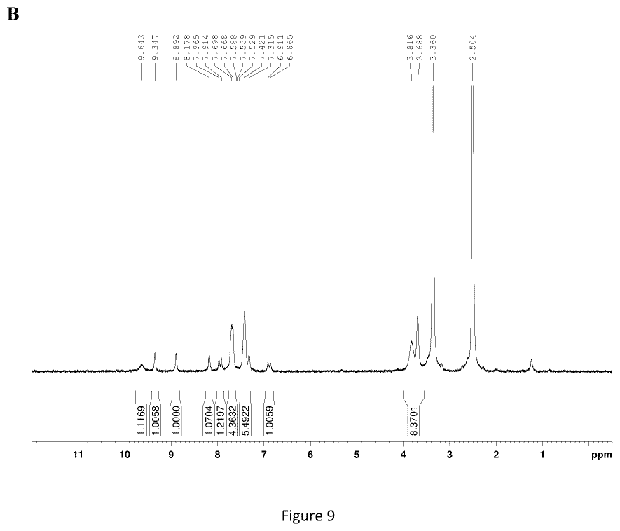

The mass spectrum and nuclear magnetic spectrum of compound L42 (C52M) are shown in FIG. 9.

### EXAMPLE

### Example 1: Vimentin-Binding s-Triazine Derivatives, Such as C50, Alter the Spatial Distribution and Fluidity of Vimentin in Cells

### Materials and Methods

Chemical synthesis: All reagents used for synthesis are at or above chemically pure grade. The final product was purified by column chromatography and characterized by ¹H NMR, ¹³C NMR, high-resolution MS and HPLC. The purity is equal to or higher than 95%.

Cell culture: Human cancer cell lines were obtained from the Institute of Cell Research, Chinese Academy of Sciences or ATCC. Cells were cultured in an incubator with 5% CO2 in RMPI1640 supplemented with 10% fetal bovine serum, 2 mM L-glutamine and 1× penicillin-streptomycin (100 IU/ml-100 μg/ml).

Immunofluorescence microscopy: Cells were cultured in 4-well coverslips, treated with DMSO or C50 for 24 h, fixed with 2% paraformaldehyde at 37° C. for 15 minutes, permeabilized in 0.2% Triton X-100 PBS at room temperature Wash for 15 min, wash 3 times with 0.05% Triton X-100 TBS, block with 10% normal goat serum in 0.05% Triton X-100 TBS, then react with mouse anti-VIM antibody (Sigma, 1:200) at room temperature Incubate for 3 h. Cells were washed 3 times with 0.05% Triton X-100 in PBS, and then incubated with Cy TM3-conjugated anti-mouse IgG antibody (Jackson, 1:300) for 1 h at room temperature. Nuclei were stained with DAPI for 5-10 min. Immunofluorescence was observed using a FV1000 (Olympus) confocal microscope.

Fluorescence recovery after photobleaching (FRAP): Cells were cultured on 4-well coverslips and transiently transfected with pEGFP-Vim expression vector using Lipofectamine 2000 (Invitrogen) following the manufacturer's instructions. Twenty-four h after transfection, cells were treated with DMSO or C50 for 24 h. FRAP experiments were performed using a confocal microscope system (Fluoview 1,000; Olympus). For photobleaching, the region of interest (ROI) was irradiated with 60% laser power (488 nm) for 2 s. Images were taken every 30 s for 5 min to monitor fluorescence recovery. Fluorescence intensities were corrected for sample background at each time point. After data collection, the fluorescence intensity of ROI was extracted by ImageJ software.

### Results and Conclusions

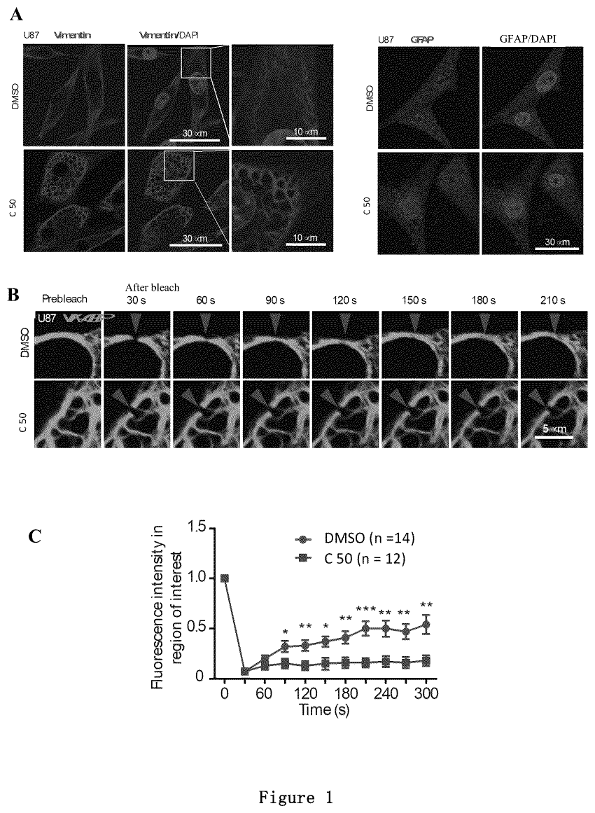

To detect possible changes in the structure of vimentin intermediate filaments in cells treated with compound C50 in vitro, confocal microscopy was used to observe immunofluorescence. Imaging revealed a significant reorganization of the vimentin network in cells with a vacuolated phenotype after C50 treatment. In control cells, vimentin filaments are arranged in bundles parallel to the longitudinal axis of the cell and are more enriched in the peripheral and distal regions of the cell than in the central region. In C50-treated cells, vimentin filaments retract and form a complex cellular network that wraps vesicles in the perinuclear region (FIG. 1, A, left panel). In order to rule out that this change is not specific to vimentin, we also detected another protein, GFAP, which belongs to the type III intermediate filament family like vimentin in U87 glioma cells. We observed no structural changes in this intermediate filament protein (FIG. 1, A, right panel). Thus, there is a specific link between C50-induced changes in cellular phenotype and changes in the spatial distribution of vimentin intermediate filaments.

We further assessed whether the structural changes of vimentin filaments caused by C50 affect its protein function. Given that the vesicles are confined within the vimentin network, it is suggested that compound C50 may influence vimentin mobility. We utilized fluorescence recovery after photobleaching (FRAP) technique to quantify the two-dimensional lateral diffusion of vimentin-GFP in cells treated with or without compound C50. Indeed, in C50-treated cells, the time required to recover fluorescence in the 0.5 m diameter ring-shaped photobleached region was significantly delayed (FIGS. 1, B and C), suggesting the C50-bound vimentin had a decreased mobility or an increased rigidity, thus hindering the transport and further disposal of intracellular vesicles.

In conclusion, binding of the s-triazine derivative C50 to vimentin alters the organization of this intermediate filament and reduces the fluidity of vimentin filaments within cells.

### Example 2: Vimentin-Binding s-Triazine Derivatives, Such as C52, Inhibit Endocytosis and Endosomal Transport

### Materials and Methods

Transfection: HEK293T cells (purchased from the Type Culture Collection Center of the Chinese Academy of Sciences, Shanghai) were seeded into 96-well plates, and reached about 70% confluence after overnight culture. Cells were transfected with 0.6 g pMAX-GPF (Lonza) plasmid DNA per well using LipoMAX (Invitrogen) according to the manufacturer's manual.

Before, after or during the whole process of transfection, compound C52 was added to the medium to test its inhibitory effect on liposome transfection efficiency. Detailed time points are described in the table below

| Group | Time of C52 treatment |
|---|---|
| Before transfection | 2 h before transfection, removed upon transfection |
| After transfection | 4 h after transfection, starting from media change |
| Entire period | Starting from 2 h before transfection |

C52 was tested at concentrations of 0.01, 0.0316, 0.1, 0.316, 1, 3.16, 10 μM. Each treatment was performed in 6 replicate wells (N=6). The medium containing the transfection reagent was removed 4 h after transfection and replaced with fresh medium. The culture plate was then placed in the Incucyte System (Essen Biosciences) for live imaging under a 10× objective lens. The fluorescence intensity (GFP signal) of each well was captured every 2 h. After 48 h, the luciferase activity in the cells was measured.

### Results and Conclusions

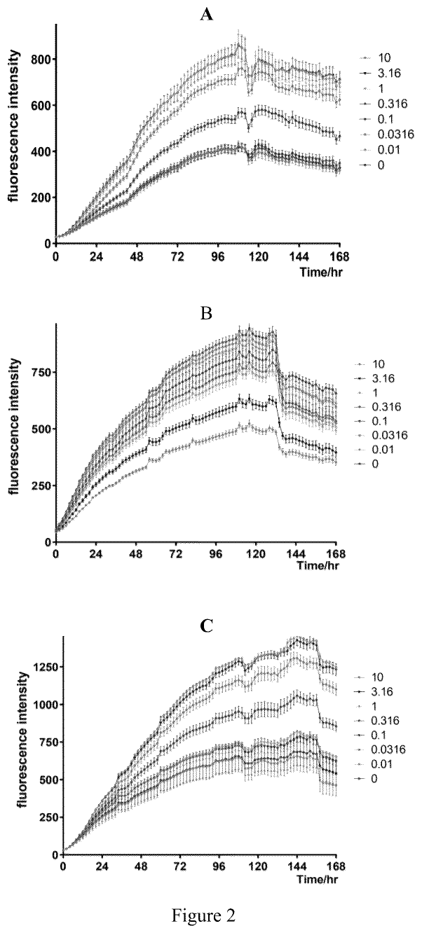

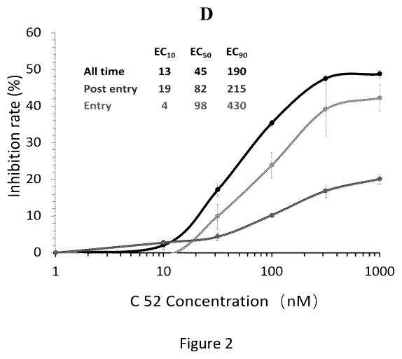

To differentiate which cellular processes were affected by compound C52, liposome-mediated transfection was performed. Cells were treated with compounds at different times during the transfection process, before, after or throughout the transfection process, representing the entry phase (endocytosis), the de-membrane phase (endosomal trafficking) and the overall process. When compound C52 was present throughout the transfection, the rate of increase in GFP fluorescence intensity was slower in C52-treated cells than in vehicle-treated cells (0 μM). The inhibitory effect of C52 on GFP expression was dose-dependent in the dose range from 0.01 to 0.316 μM (FIG. 2, A). The maximum inhibition rate of about 50% can be reached at 0.316 μM, and the maximum inhibition rate is maintained between 0.316 and 10 μM. When cells were treated with compound C52 4 h after transfection, the inhibition of GFP expression reached a maximum of approximately 40% at 0.316 μM, with only a slight increase between 0.316 and 10 μM (FIG. 2, B). When cells were treated with compound C52 just before infection, GFP was inhibited by approximately 15% at 0.316 μM and further increased to a maximum of approximately 40% at 10 μM (FIG. 2, C).

The results indicate that compound C52 can effectively inhibit liposome-mediated transfection, and the maximum inhibition rate is about 50%, which is mainly by inhibiting endosomal trafficking, and to a lesser extent inhibiting endocytosis. Compound C52 inhibited endocytosis, endosomal trafficking and the whole process with EC50 of 98, 82 and 45 nM, respectively (FIG. 2, D).

### Example 3: Vimentin-Binding s-Triazine Derivatives, Such as C45, C52, C69A, C69B and C52M, Inhibit the Release of Exosomes from Liver Cancer Cells

### Materials and Methods

Cells: The liver cancer cell line Huh7 was obtained from the JCRB cell bank and cultured with DMEM medium supplemented with 10% fetal bovine serum (FBS), penicillin (100U/ml) and streptomycin (100 ug/ml). When exosome purification was performed on supernatants from cell cultures, FBS in all media was replaced with exosome-depleted FBS (Evomic Science, Sunnyvale, CA).

Lentivirus: The exosome reporter plasmid pLenti-PGK-Luc-Exo was constructed by Evomic Science scientists. Following company standard protocols, high titers of lentiviral particles were produced in 293T. Huh7 cell line was infected overnight with the pLenti-PGK-Luc-Exo lentiviral particles at MOI 10:1. After three passages, stably transfected cells were used to isolate exosomes in the culture supernatant. Luciferase activity in exosomes was used to quantify exosomes in the supernatant.

Drug treatment: Compounds C52, C69A, C69B, C45 and C52M were dissolved in DMSO to make stock solutions (10 mM or 20 mM). The Huh7 cell line harboring pLenti-PGK-Luc-Exo was seeded into 96-well plates (clear white or black) and grown overnight to 90% confluency. Cells were treated with the indicated concentrations of compounds. The compound GW4869 (#D1692, Sigma, St Louis, MO), known to inhibit exosome release, was used as a positive control to ensure the quality of the experiment. Cells treated with 0.1% DMSO served as a negative control. Two days after treatment, the supernatant from each well in the 96-well plate was collected separately. At this point, cells on the 96-well plate were incubated with 10% PrestoBlue cell viability reagent (ThermoFisher Scientific, Santa Clara, CA) in cell culture medium for 30 min. Cell viability in each well was assessed by measuring fluorescence in a TECAN Infinite M100 (TECAN, San Jose, CA).

Exosome release inhibitors were screened using the ExoHTPTM platform: the supernatant (approximately 98 μl) from each well was collected and centrifuged at 300 g for 10 min to remove cells, then at 2200 g for 30 min to remove apoptotic bodies and large extracellular vesicles. The processed supernatant was used to isolate exosomes by Evomic Science's ExoEZTM Exosome Isolation Kit (#ExoCC50, Sunnyvale, CA). Briefly, 80 μl of the treated supernatant was mixed well with 40 μl of buffer P1, 40 μl of buffer P2 and 1 μl of buffers D and F, respectively. After thorough mixing, the supernatant was centrifuged at 2200 g, 4° C. for 30 minutes. After removing the supernatant (140 μl), 140 μl of Wash Buffer W was added to the exosomes, followed by centrifugation at 2200 g for 10 min. After removing 140 μl of the supernatant, the exosome pellet was well suspended in 130 μl of PBS. Luciferase activity in 50 μl of exosome suspension was measured in a TECAN Infinite M100 (TECAN, San Jose, USA) with the Promega Renilla Luciferase Assay Kit (#E2810, Madison, WI).

### Results and Conclusions

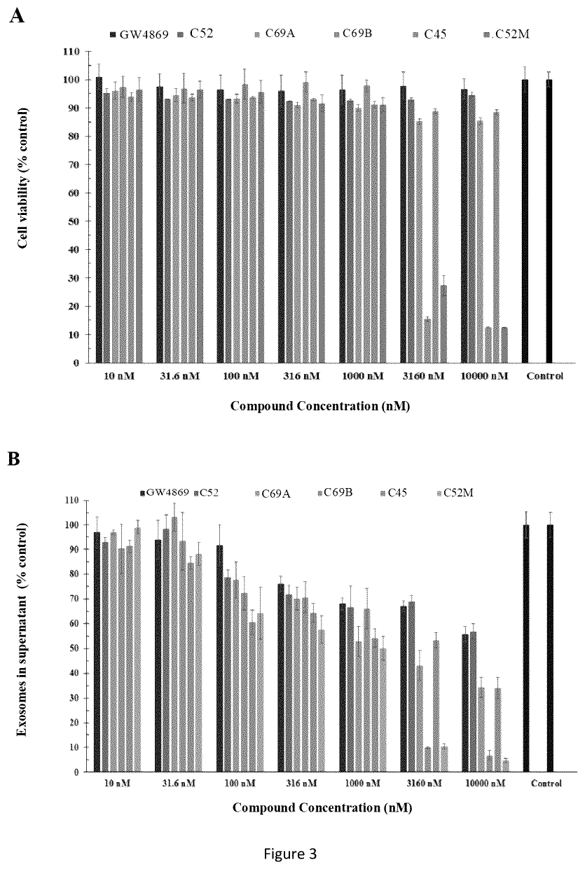

Cell survival: After two days of treatment with compounds GW4869, C52, C69A and C45, the survival rate of liver cancer Huh7-NC12 was about 90% (FIG. 3, A). However, when cells were treated with C69B and C52M, Huh7-NC12 showed significant cytotoxicity (survival rate was less than 30%) at concentrations exceeding 3160 nM. These data indicated that compounds C52, C69A and C45 were not toxic even at high concentrations (>3160 nM), while compounds C69B and C52M were cytotoxic to Huh7-NC12 cells at high concentrations (>3160 nM).

Exosome release: When Huh7-NC12 was treated with various compounds, exosomes in the supernatant were purified using the ExoEZTM Exosome Isolation Kit (Evomics), and then quantified by measuring luciferase activity. A known exosome release inhibitor GW4869 was used as a positive control. As shown in FIG. 3, B, all compounds had the ability to inhibit exosome release. Exosome inhibition was shown to be dose-dependent. In particular, C52 inhibited exosome release at a level comparable to that of the known exosome inhibitor GW4869, whereas C69A and C45 had stronger inhibitory effects than GW4869.

### Example 4: Vimentin-Binding s-Triazine Derivatives, Such as C52, Inhibit the Release of Exosomes from Different Types of Cancer Cells In Vitro

### Materials and Methods

Human non-small cell lung cancer A549 cells and human pancreatic cancer PANC-1 cells were purchased from the Chinese Academy of Sciences Stem Cell Bank (Shanghai, China). Cells were cultured at 37° C. in RPMI1640 medium (humidified environment with 5% CO2) containing 10% fetal bovine serum (Sigma, USA), 100 U penicillin and 100 μg/ml streptomycin (Gibco, USA).

Purification, characterization, and analysis of exosomes: To purify exosomes from cell culture supernatants, cells were cultured for 3 days in DMEM medium supplemented with 10% exosome-free FBS prepared by ultracentrifugation of regular FBS at 120,000 g for 16 h. Cell culture supernatants were collected and centrifuged at 500 g for 5 min and then at 2,000 g for 20 min to remove dead cells and cell debris. Then, large vesicles were removed by centrifugation at 12,000 g for 30 minutes. Crude exosomes were pelleted by ultracentrifugation of the supernatant at 110,000 g for 70 min (Backman Ti70), washed with PBS and filtered (0.2 μm). The pellet was then suspended and ultracentrifuged again at 110,000 g for 70 minutes. All ultracentrifugations were performed at 4° C. The size and number of exosomes were analyzed using an LM10 nanoparticle tracking system (NanoSight) equipped with a blue laser (405 nm).

Transmission electron microscope (TEM) observation: The morphology of exosomes prepared from the culture supernatant was observed by transmission electron microscope (TEM). Exosome pellets were fixed overnight at 4° C. in 2% glutaraldehyde. Load a drop of 10 μL of the exosome suspension onto the Formvar/Carbon-coated TEM copper grid and let it stand for 20 min. Drain excess fluid. Samples were fixed with 1% uranyl acetate for 5 min, washed 8 times with double distilled water, and dried under incandescent lamp for 10 min, then examined with Tecnai G2 Spirit Bio TWIN transmission electron microscope (FEI, Hillsboro, USA, working voltage 120 kV).

### Results and Conclusions

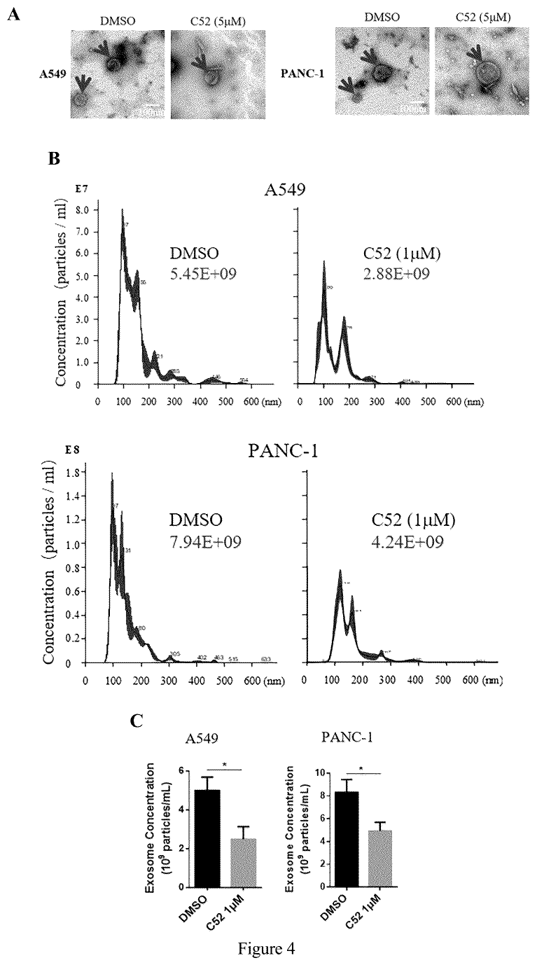

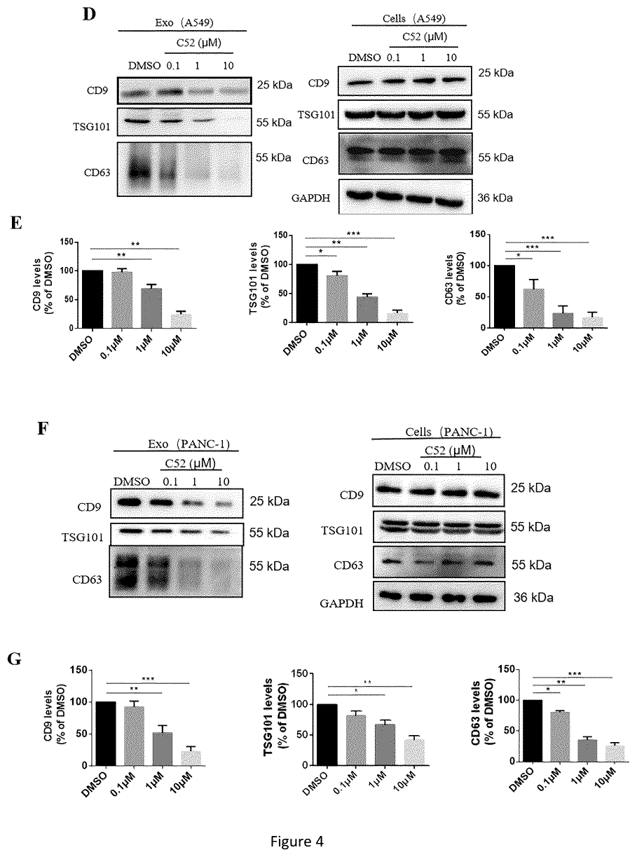

Exosomes in the cell culture supernatant were purified and quantified from human non-small cell lung cancer A549 cells and human pancreatic cancer PANC-1 cells treated with compound C52. The exosomes obtained by a classic exosome purification method through three rounds of centrifugation had a typical disc-like structure morphology, with diameters ranging from 50-150 nm (FIG. 4, A). Nanotracking analysis showed that exosomes were significantly reduced in the culture supernatants of A549 cells and PANC-1 cells treated with compound C52 (FIGS. 4, B and C). Western blotting also confirmed that compound C52 treatment resulted in a significant reduction in the content of exosomal markers in the prepared exosome samples (FIGS. 4, D, left panel; E; F, left panel; and G). In contrast, compound C52 treatment did not reduce intracellular exosome levels (FIGS. 4, D, right panel; and F, right panel), suggesting that compound C52 blocked exosome release from cells, rather than preventing exosomes produced in the cell.

### Example 5: Vimentin Regulates the Generation and Release of Exosomes; Vimentin-Binding Triazine Derivatives, Such as C52, Inhibit Exosome Release from Human Glioma Cells

### Materials and Methods

CRISPR Design and Vimentin Gene Editing: Plasmid pB-TRE-NLS-linker-Cas9-ZF-IRES-hrGFP-Blasticidin was used for the doxycycline-inducible expression of human codon-optimized Cas9, and plasmid pGL3-U6-2sgRNA-ccdB-EF1a-Puromycin, for the expression of vimentin gene-specific sgRNA. With the aid of the CRISPR design tool (http://crispr.mit.edu/), two guide strands were designed targeting two regions of exon 1 (Exon 1) of the human vimentin gene (target site 1: GTCCTCGTCCTCCTACCGC, SEQ ID NO: 1; target site 2: CGGGCTCCTGCAGGACTCGG, SEQ ID NO: 2). The sgRNA coding sequence was cloned into the sgRNA expression vector at the BsmBI site. The plasmid expressing Cas9 was transfected into U87 cells by Lipofectamine 2000 (Invitrogen) and screened by blasticidin. Stable cell lines were treated with doxycycline for 12 h to induce Cas9 expression, then transfected with a vector expressing vimentin sgRNA, and transduced cells were selected with Puromycin 24 h later. Collect pooled clonal cells. Respectively by PCR (knockout: forward primer ATGTTCGGCGGCCCGGGCAC (SEQ ID NO:3), reverse primer 15 AGGAGCCGCACCCCGGGCACG (SEQ ID NO:4); for wild type: forward primer GAGGGGACCCTCTTTCCTAA (SEQ ID NO:5), reverse primer GGTGGACGTAGTCACGTAGC (SEQ ID NO:6)) and Western blotting confirmed deletion of the target gene region and deletion of vimentin protein.

Western blotting: Exosomes from cultures in different conditions were collected and purified by a classical differential centrifugation protocol, and then lysed with PMSF-containing RIPA buffer (Beyond Biotechnology, Shanghai, China). The lysate was centrifuged to remove insoluble material. The concentration of protein was determined by enhanced BCA protein assay kit (Beyontian Biotechnology, Shanghai, China). 40 g of proteins were separated by sodium dodecyl sulfate-polyacrylamide gel electrophoresis (SDS-PAGE) and transferred to a polyvinylidene difluoride membrane (Merck Millipore). Membranes were blocked with 5% milk for 1 h and then incubated with primary antibodies overnight at 4° C. Membranes were washed 3 times and then incubated with secondary antibodies for 1 h at room temperature. Bands were detected by Gel Imaging System (Bio-Rad, USA) and measured by Image J. The primary antibodies used were rabbit anti-β-actin, GAPHD, CD63, CD9, TSG101 and vimentin (Abcam, UK).

### Results and Conclusions

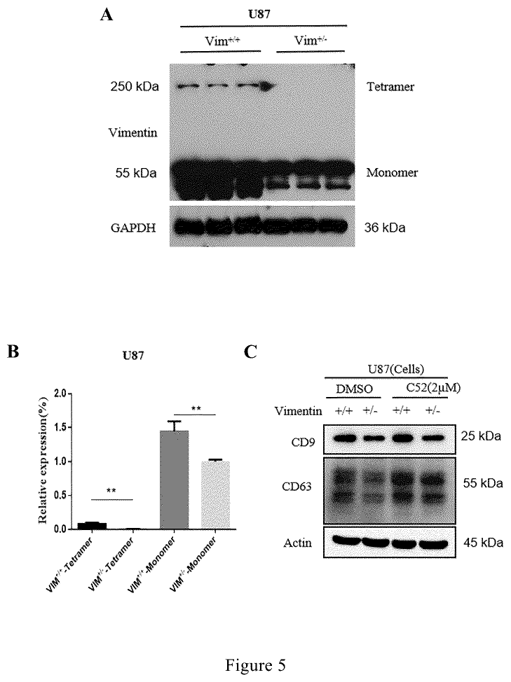

To determine whether the blockage of exosome release by C52 was a direct result of its binding to vimentin, we attempted to knockout the vimentin gene from human glioma U87 cells using the CRISPR-cas9 system. We were not able to obtain Vim−/− clones, all clones we got were Vim+/− with only one allele knocked out. The lack of viable Vim−/− clones may indicate that vimentin plays a key role in this particular cancer type. Western blotting showed that compared with U87 Vim+/+ cells, U87 Vim+/− cells had lower intracellular vimentin content (FIGS. 5, A and B), and correspondingly, intracellular exosome content was also lower (FIG. 5, C-E), showing that vimentin promotes the formation of extracellular exosomes. C52 treatment did not reduce the amount of exosomal marker CD9 (FIGS. 5, C and D) or CD63 (FIGS. 5, C and E) inside the U87 Vim+/+ cells or U87 Vim+/− cells. These results suggest that, unlike vimentin knockdown, C52 treatment does not reduce the number of intracellular exosomes (or ILVs) that have not been released. Western blot (FIG. 5, F) and quantification (FIGS. 5, G and H) showed that vimentin knockdown and C52 treatment both reduced the extracellular levels of exosomal markers CD9 and CD63 compared with U87 Vim+/+ (FIG. 5, F-H); in vimentin-knockdown U87 Vim+/− cells, compound C52 treatment led to a further reduction of CD9 in the cell culture medium (FIGS. 5, F and G). These results suggest that compound C52, which binds to vimentin, mainly blocks the release of exosomes from intracellular to extracellular.

### Example 6: Vimentin-Binding Compound C52 Significantly Inhibits the Migration and Invasive Growth of Cancer Cells In Vitro, and Reduces the Number of Circulating Exosomes In Vivo

### Materials and Methods

Wound healing assay: Cells were seeded in 6-well plates containing complete medium and cultured at 37° C. in a humidified air environment containing 5% CO2. After the cells reach 90% confluency, make a vertical wound using a 200 μL plastic pipette tip. Then, the cells were washed 3 times with PBS, and then incubated with serum-free RPMI-1640 medium containing different concentrations of C52 for 24 h. The wound area and migrated cells within the wound area were photographed using a phase-contrast microscope. The wound area was measured using Image J software.

Cell invasive growth assay: Inoculate 1×10E4 cells suspended in 100 μL serum-free RPMI-1640 medium into each upper chamber of a 6-well Transwell pre-coated with Matrigel and added with DMSO or C52 (Cat.3422, Corning, NY, USA). in different concentrations. Add 600 μL of RPMI-1640 containing 10% FBS to the bottom chamber of each well. Cells in transwell plates were cultured for 24 h. Cells on the upper surface were gently wiped with a moistened cotton swab, while cells that passed through the Matrigel to the lower surface of the membrane were fixed with paraformaldehyde and stained with 0.1% crystal violet. These cells on the lower surface were considered invasive and counted in 5 random fields under an inverted microscope. The number of invasive cells was counted by Image J software.

In vivo experiments: 6- to 8-week-old BALB/c mice (18 to 20 g) were purchased from Beijing Life River Experimental Animal Technology Co., Ltd. (Beijing, China) and housed at the Experimental Animal Center of Nanjing University of Traditional Chinese Medicine. Animals were maintained at 22±2° C. on a regular 12h/12h light-dark cycle with free access to food and water. All animal experiments were approved by the Ethics Committee of Nanjing University of Traditional Chinese Medicine. Animal care was provided according to IACUC-approved guidelines.

Three mice per group received 100 mg/kg of C52 or the same volume of vehicle once a day for 14 consecutive days. Twenty-four hours after the last administration, the mice were anesthetized with pentobarbital sodium, from which blood samples were collected, and then the mice were euthanized. Plasma exosomes were prepared by sucrose density gradient centrifugation and analyzed by Western blotting.

Isolation of exosomes from mouse plasma: To obtain plasma for exosome isolation, blood was centrifuged at 500 g for 5 min at 4° C.; 2000 g for 15 min; and treated with a solution of 10000 g for 20 min to remove cells, debris and large vesicles. The crude exosome pellet was resuspended in 60 ml of cold PBS (Invitrogen), and then the exosome suspension was centrifuged at 100,000 g for 70 min at 4° C. For sucrose density gradient centrifugation, mix the washed exosome pellet with 2 mL of 2.5 μM sucrose solution and add to a sucrose step gradient column (10 2 ml sucrose gradients from 2 μM to 0.4 μM using 20 mM HEPES as diluent). The sucrose step gradient was centrifuged at 200,000 g (Backman Ti70) for 16 h at 4° C. Fractions were collected and recentrifuged in cold PBS at 100,000 g for 70 min at 4° C. All fraction pellets were resuspended in 30 μl lysis buffer before further western blot analysis.

### Results and Conclusions

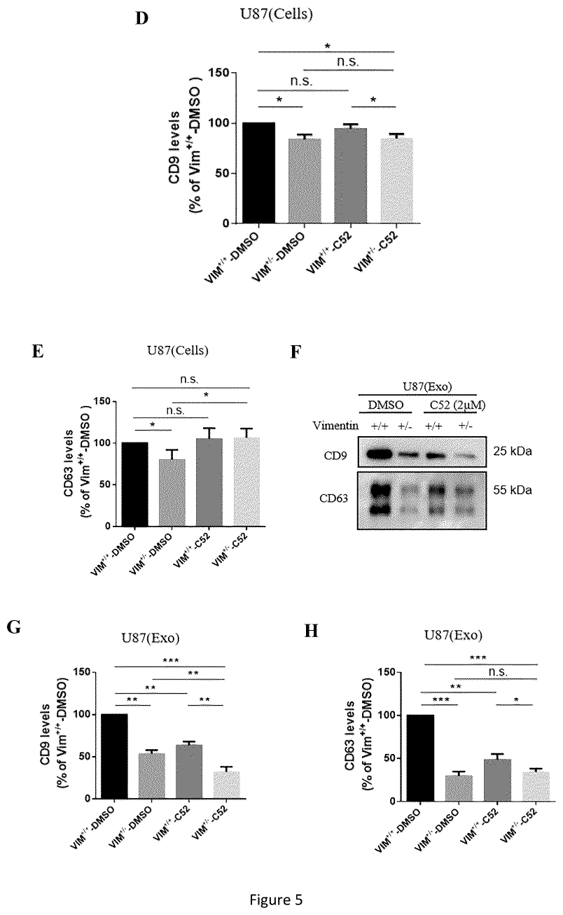

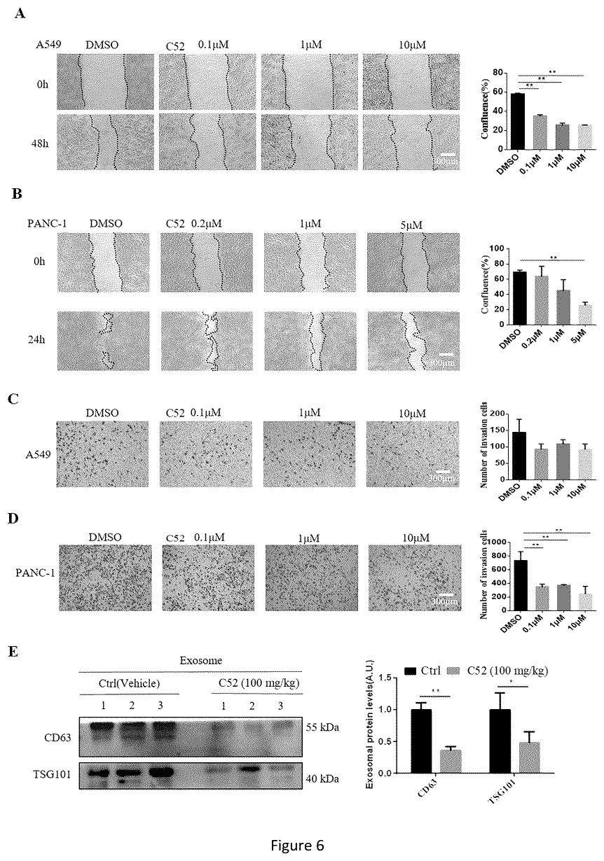

Vimentin is known to promote cell migration, while exosomes increase cell mobility (Ivaska, J. et al., Novel functions of vimentin in cell adhesion, migration, and signaling. Exp. Cell Res. 313, 2050-2062, 2007; Hoshino, A. et al. Tumor exosome integrins determine organotropic metastasis. Nature 527, 329-335, 2015). We investigated whether compounds that bind to vimentin affect the migration and invasion of cancer cells. Wound healing assays showed that in lung cancer A549 cells (FIG. 6, A) and pancreatic cancer PANC-1 cells (FIG. 6, B), the closure rate of the gap size was significantly decreased over time after C52 treatment in a dose-dependent manner. Transwell analysis showed that C52 hindered the migratory ability of both types of cancer cells, which was more pronounced in PANC-1 cells (FIG. 6, D) compared with A549 cells (FIG. 6, C). Since PANC-1 cells migrate faster than A549 cells, the compound C52, which binds to vimentin, had a greater inhibitory effect on the more aggressively growing cancer cells.

To confirm the activity of C52 in vivo, we administered the compound to mice daily for 14 days by gavage. Exosomes were purified from mouse plasma, and exosomal markers were quantified by Western blot. In exosomes isolated from C52-treated mice, both CD63 and TSG101 were significantly decreased compared with exosomes isolated from vehicle-treated mice (FIG. 6, E). It can be concluded that oral administration of C52 in mice can reduce the number of circulating exosomes.

### Example 7: Vimentin-Binding Triazine Derivatives, Such as C52, Strongly Inhibit the SARS-CoV2 Spike Protein and Human ACE-Mediated Lentiviral Infection In Vitro

### Materials and Methods

HEK293T cells expressing human ACE-2 receptor were infected with Pseudovirus-2019-nCoV-GFP-IRES LUC (Fubaiao, Suzhou) containing the SARS-CoV2 spike protein on the outer membrane: 1×10E4 cells per well were seeded into 96-well plates, the cells reached 40% confluency the next day (approximately 2×10E4 cells) and were used for virus infection. Virus titers were determined by the supplier as 2×10E7 TFU/mL. For cell infection, use 2×10E4 (MOI=1), 2×10E5 (MOI=10), and 2×10E6 (MOI=100) TFU/mL virus per well in 100 μL of medium.

C52 was present in the medium throughout the infection. The tested concentrations of C52 were: 0.01, 0.0316, 0.1, 0.316, 1, 3.16, 10 μM. Cells treated with NH4Cl were used as a positive inhibition control. Each treatment was performed in triplicate wells (N=3).

After overnight post-infection incubation (approximately 16 h), the virus-containing medium was removed and replaced with fresh medium. The culture plate was then placed in the Incucyte System (Essen Biosciences) for live imaging under a 10× objective lens. The fluorescence intensity (GFP signal) of each well was captured every 2 h. After 48 h of imaging, intracellular luciferase (Luciferase) activity was measured, and the luminescent signal of each well was measured by a BioTek Synergy 4 plate reader.

### Results and Conclusions

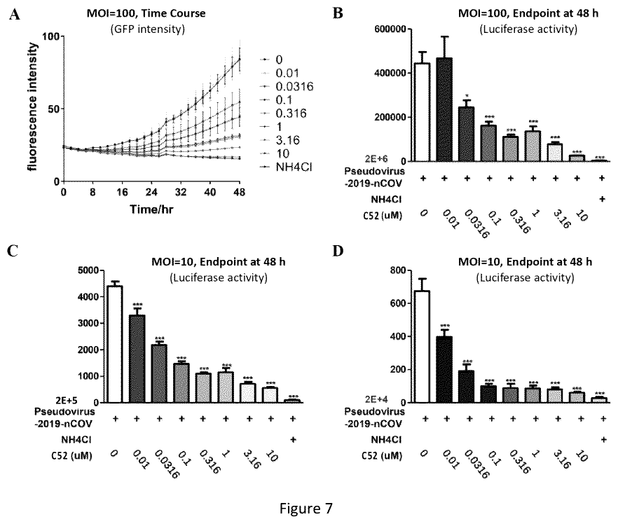

When C52 was present throughout the infection process, the GFP signal increased more slowly in C52-treated cells than in untreated cells (FIG. 7, A). This inhibitory effect was also dose-dependent, and the inhibition by 10 μM of C52 was as strong as that by the positive control (NH4Cl-treated group). End-point luciferase assays also showed similar results. Despite the use of different virus titers, even at high virus infection doses (MOI=100), C52 treatment showed dose-dependent inhibition of pseudovirus infection with IC50 less than 50 nM (FIG. 7, B-D), indicating that C52 Strongly inhibits the infection of pseudovirus-2019-nCOV.

### Example 8: Vimentin-Binding s-Triazine Derivatives, Such as C52, Significantly Improve Clinical Symptoms and Lung Damage in Aged Mice with SARS-CoV2 Infection

### Materials and Methods

Virus: SARS-CoV-2 MA10 (a mouse-adapted virulent mutant) was generated from a synthetic sequence of recombinant Washington strain in Baric's lab at the University of North Carolina (Leist, et al. A Mouse-Adapted SARS-CoV-2 Induces Acute Lung Injury and Mortality in Standard Laboratory Mice. Cell 183:1070-1085.e]2, 2020). Viruses were kept at low passage (P2-P3) to prevent the accumulation of other potentially confounding mutations.

Animals: Aged (11- to 12-month-old) female BALB/c mice obtained from Envigo (retired breeder) were intranasally infected with 10E3 PFU of the virus SARS-CoV-2 MA10. The gene sequence and titer of the virus were verified. 50 μl of the virus preparation in DMEM diluted with PB S to the desired concentration for the inoculation. Animals were acclimatized for 7 days in a BSL-3 environment with a 12h/12h light/dark cycle, 5/cage, with free access to food and water before any experiment was performed. Before infection, animals were anesthetized by intraperitoneal injection using 50 μl of a combination of 50 mg/kg ketamine and 15 mg/kg Xylazine.

Experimental design: 40 animals were evaluated in each treatment group (prophylaxis and treatment), 10 animals per dose group. See the table below.

| | | | | Euthanasia |
|---|---|---|---|---|
| Treatment | | Treatment | Infection | Day Post- |
| Arm | Dosage | Times (h) | Conditions | Infection |
| Prophylactic | Vehicle | −15, −5, +24, | 10³ PFU SARS- | 5 |
| G2 | | +48, +72, +96 | CoV-2 MA10 | |
| G3 | 3 mg/kg | −15, −5, +24, | 10³ PFU SARS- | 5 |
| | | +48, +72, +96 | CoV-2 MA10 | |
| G4 | 10 mg/kg | −15, −5, +24, | 10³ PFU SARS- | 5 |
| | | +48, +72, +96 | CoV-2 MA10 | |
| G5 | 30 mg/kg | −15, −5, +24, | 10³ PFU SARS- | 5 |
| | | +48, +72, +96 | CoV-2 MA10 | |
| Therapeutic | 3 mg/kg | +24, +48, +96 | 10³ PFU SARS- | 5 |
| G6 | | | CoV-2 MA10 | |
| G7 | 10 mg/kg | +24, +48, +96 | 10³ PFU SARS- | 5 |
| | | | CoV-2 MA10 | |
| G8 | 30 mg/kg | +24, +48, +96 | 10³ PFU SARS- | 5 |
| | | | CoV-2 MA10 | |
| Mock G1 | Vehicle | +24, +48, +96 | PBS | 5 |

Procedures: In the prophylactic arm, C52 was administered to mice −15 and −5 h prior to infection. Subsequent doses were administered QD at approximately the same time each day post-infection, starting at 24 h post-infection. Administration was via oral gavage in 125 μl. In the therapeutic arm: C52 was administered to mice starting at 24 h post-infection. Subsequent doses were administered QD at approximately the same time each day post-infection. Administration was via oral gavage in 100 μl.

The procedures conducted to monitor infection included (1) daily clinical evaluation and scoring, including body weight and disease score, and (2) animals that survived anesthesia were carried to the experimental endpoint.

Note: As the mice were continuing to lose weight and the disease scores were increasing, animals were euthanized at 5 days post-infection. Euthanasia was performed by inhalational isoflurane (drop method) and thoracotomy, with removal of vital organs (lungs). Lung tissue was taken for assessments of titer, RNA, and histology. Serum was taken for downstream analysis.

### Results and Conclusion

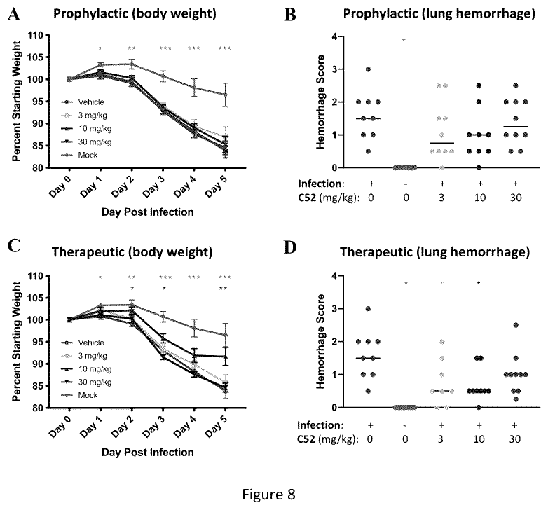

While the mock infected mice maintained a stable body weight (within ±3%), all the SARS-CoV-2-infected mice showed various degrees of body weight loss over the course of the study. There was no significant difference in body weight loss between the vehicle group and either of the prophylactic groups (3, 10, 30 mg/kg) (FIG. 8, A) or of the therapeutic groups (1, 30 mg/kg). However, the body weight loss was significantly less in the therapeutic group of mice received C52 at 10 mg/kg than in the vehicle group (FIG. 8, C).

As expected, none of the mice in the mock infected group and all of the mice in the vehicle group developed hemorrhage. The hemorrhage in the mice from prophylactic treatment groups (1, 10, 30 mg/kg) appeared to be less severe than that in mice from the vehicle group, the difference however, was not statistically significant despite the lack of hemorrhage in one mouse each in the 3 mg/kg and 10 mg/kg dose groups (FIG. 8, B). For the mice received therapeutic treatment, the hemorrhage scores were significantly less in the 3 mg/kg and 10 mg/kg dose groups than in the vehicle group (FIG. 8.D), while the difference between the 30 mg/kg dose group and the vehicle group was not significant.

In summary, the compound C52 at the low (3 mg/kg) and the mid (10 mg/kg) doses orally administered 24 h after SARS-CoV2 infection significantly reduced hemorrhage in the mouse lung. The therapeutic treatment with C52 at the mid dose also significantly ameliorated the body weight loss.

### Example 9: Vimentin-Binding s-Triazine Derivatives, Such as C50 and C52, have No Toxic Effect on Primary Human Hepatocytes and Primary Human Endothelial Cells In Vitro

Safety is the prerequisite for all therapeutic drugs, especially for chronic diseases, such as autoimmune diseases. We use primary human hepatocytes and primary human endothelial cells to show that vimentin-targeted s-triazine derivatives are not harmful to normal human cells.

### Materials and Methods

Human Hepatocyte Culture: Cryopreserved plateable human hepatocytes were purchased from BioIVT (Westbury, NY) and cultured in hepatocyte culture media, which contains F12/DMEM with 1% FBS, 1% human serum AB type, lx lipid-rich Albumin2 (Gibco, AlbuMAX, #11020-013); 1×B27 (Gibco, #17504044,); 1×N2 supplement (Gibco, #17502048), 10 mM nicotinamide; 1×ITS Universal Cell Culture Supplement Premix (BD, #354351), 10 g/ml ascorbic acid (Sigma, A4403); 5 ng/ml HGF, 20 ng/ml EGF; 3 μM CHIR (Tocris, #4423); 3 μM A8301 (Tocris, #2939/10). After seeding 100,000 cells on each well of collagen IV-coated 24 well plate for overnight, hepatocytes were treated with the indicated concentration of compounds in fresh hepatocyte culture media.

HUVEC Culture: Pooled human umbilical vein endothelial cell (HUVEC) was purchased from Lonza. 50,000 of HUVEC (Passage #6) were seeded on each well of 1% gelatin-coated 48 well plate, with culture media EGM2 bullet kit (Lonza). HUVECs were treated with the indicated concentration of compounds in fresh HUVEC culture media.

Measurement: All cells were cultured in a humidified, 5% carbon dioxide, 95% air incubator at 37° C. After overnight culture, cells were then treated with the indicated concentration of compounds. After 3 days of treatment, cell morphology was recorded. Cells were washed with PBS once and then 500 μl of fresh culture media containing 10% of PrestoBlue (ThermoFisher Scientific) were added to each well. After 50 min of incubation, fluorescence in each well was measured in fluorescent reader (Molecular Devices SpectraMax). Fluorescent unit in each well was normalized to that of control hepatocytes or HUVEC, represented as cell viability which displayed in mean±SD.

### Results and Conclusion

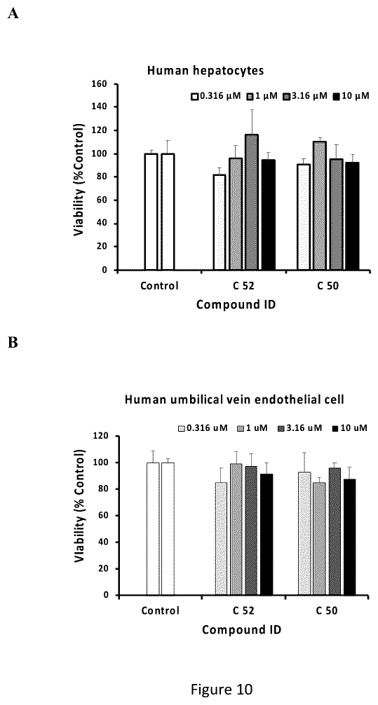

At the highest concentration of 10 μM, both C50 and C52 compounds had no toxic effects on human hepatocytes (FIG. 10, A) and human umbilical vein endothelial cells (FIG. 10, B).

### Example 10: Vimentin-Binding s-Triazine Derivatives, Such as C52, have No Effect on the Growth of Various Types of Cancer Cells and the Major Signal Transduction Pathways Associated with Cancer Cell Growth In Vitro

In the previous example, we demonstrated that the vimentin-binding s-triazine derivatives had no harmful effect on normal human cells. In order to further confirm that the vimentin-targeting s-triazine derivatives do not directly cause cytotoxicity or do not have mechanism to directly affect cell growth, we tested the compound C52 in a variety of cancer cells.

### Materials and Methods

Cell culture: Human non-small cell lung cancer A549 cells, human pancreatic cancer PANC-1 cells, human glioma U87 cells, human liver cancer HUH7 cells and SMMC-7721 cells, and human gastric cancer AGS cells were purchased from ATCC or Stem Cell Bank of Chinese Academy of Sciences (Shanghai, China). Cells were cultured in RPMI1640 medium containing 10% fetal bovine serum (Sigma, USA), 100 U penicillin and 100 μg/ml streptomycin (Gibco, USA) at 37° C. in a humidified atmosphere containing 5% CO2.

Protein microarray analysis: Cell lysates were extracted from A549, and PANC-1 cells treated with 3 μM C52 or an equal volume of DMSO. A total of 200 g of lysate protein was run on one Human XL Tumor Array (R&D Systems, Abingdon, UK). Followed the manufacturer's instructions exactly. The protein chip membrane was covered with plastic film and exposed to X-ray film for 8 minutes. Optical density on exposed X-ray film (hu.q, HQ-320XT, China) was quantified using a transmission mode scanner (EPSON, Beijing, China) and analyzed using Image J software.

### Results and Conclusions

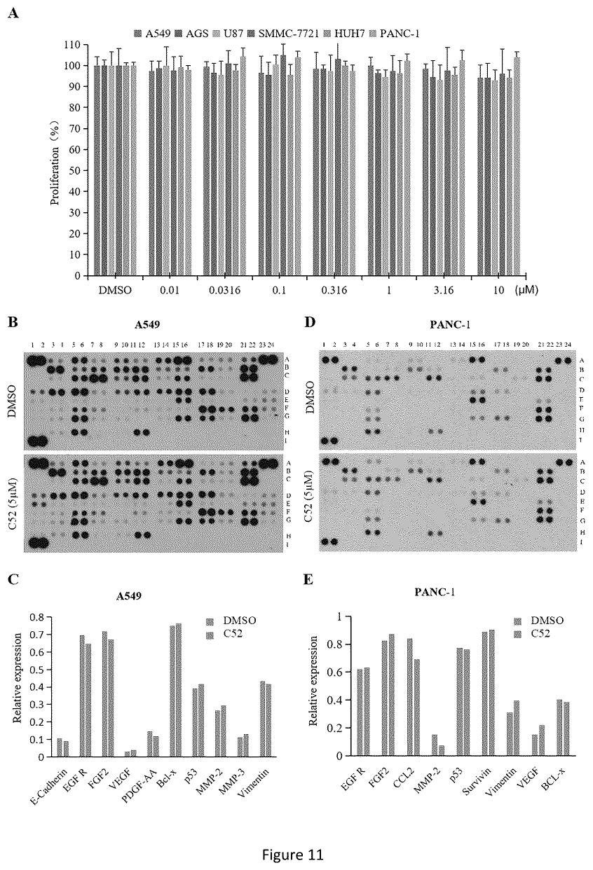

To test whether the phenotypic changes induced by the vimentin-binding compound C52 lead to inhibition of cancer cell growth, A549, AGS, U87, SMMC-7721, HUH7 were treated with different concentrations of the compound. The highest concentration of compound C52 was 10 μM. At any concentration, C52 had no effect on the growth of these cells, neither growth-inhibitory nor growth-promoting (FIG. 11, A).

To determine whether the phenotypic changes induced by vimentin-binding compounds in cancer cells were associated with any oncogenic signaling pathways, proteome profiling was performed using protein microarrays. Using a protein array consisting of 84 selected human cancer-associated proteins, little or no effect of compound C52 was found on protein levels of vimentin and others signaling molecules involved in cell proliferation in A549 (FIGS. 11, B and C) and PANC-1 (FIGS. 11, D and E) cells.

Therefore, the binding of s-triazine derivative C52 to vimentin has no direct inhibitory or promoting effect on the growth of normal cells and tumor cells, and has no effect on the main signaling pathways related to cell growth.

### Example 11: In Vivo, Vimentin-Binding s-Triazine Derivatives, Such as C52, not Only Enhance the Function of Treg Cells, but Also Promote the Regeneration of Treg Cells after their Depletion

It is known that vimentin can restrain the function of regulatory T cells (Treg), and the vimentin-binding s-triazine derivatives negate the action of vimentin on Treg cells. Treg cells have strong plasticity. Functional Treg cells in vitro may completely lose their proper function in vivo (Sakaguchi, et al. The plasticity and stability of regulatory T cells. Nat Rev Immunol. 13:461-7, 2013; Qiu, et al. Regulatory T Cell Plasticity and Stability and Autoimmune Diseases. Clin Rev Allergy Immunol. 58:52-70, 2020). Therefore, the ability to activate Treg cells in vivo is the key to Treg-related therapy. We used a syngeneic mouse tumor model to demonstrate the effect of vimentin-binding s-triazine derivatives on Treg cells in vivo.

### Materials and Methods

BALB/c mice were subcutaneously inoculated with 1×10E6 CT26 cells. Mice were randomly assigned to each treatment group. The tumor size was measured with a caliper, and the tumor volume was calculated by the following formula: V=length×width 2×1/2. Tumor-bearing mice were orally administered with vehicle or 100 mg/kg C52, QD. On the 13th day after tumor inoculation, vehicle- and C52-treated mice were given a single intraperitoneal injection of low-dose (50 mg/kg) cyclophosphamide (Sigma-Aldrich). Mice were euthanized before tumors reached 2.0 cm in longest dimension. Spleen and lymph node samples were collected for flow cytometric analysis.

### Results and Conclusions

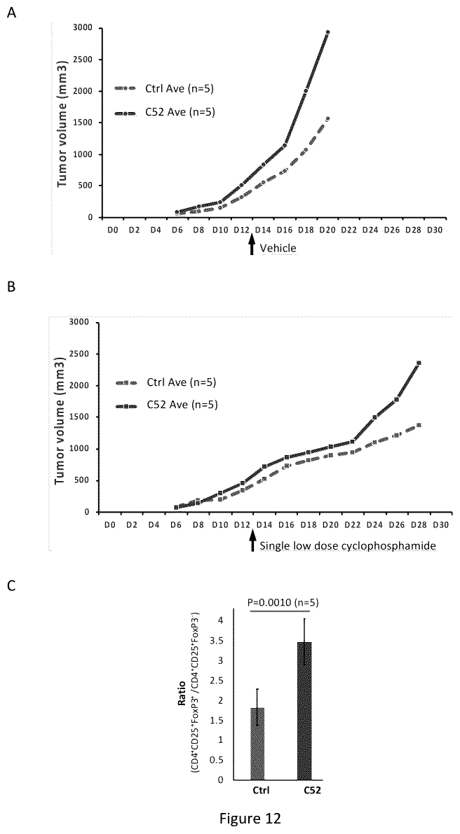

Vimentin is known to restrain Treg activity, therefore, vimentin-targeting compound C52 may activate Treg cells. Although C52 has no effect on tumor growth in vitro, its activation of Treg will be beneficial to tumor growth in vivo. To test the ability of C52 to activate Tregs in vivo, we used a syngeneic mouse CT26 tumor model. Indeed, tumors appeared to grow faster in mice treated with C52 than in vehicle-treated control mice (FIG. 12, A). To confirm that this phenomenon is due to the activation of Treg cells, at day 13 after tumor inoculation. All mice were given a single intraperitoneal injection of saline (FIG. 12, A) or a low dose (50 mg/kg) of cyclophosphamide (FIG. 12, B). It is well established that low-dose cyclophosphamide (LDCP) selectively depletes Treg cells and is not sufficient to kill cancer cells (Ghiringhelli, et al. Metronomic Cyclophosphamide Regimen Selectively Depletes CD4+CD25+ Regulatory T Cells and Restores T and NK Effector Functions in End Stage Cancer Patients. Cancer Immunol. Immunother. 56:641-648, 2007). As expected, administration of normal saline injection did not affect the effect of C52 on tumor growth in vivo (FIG. 12, A, shaded part); while on day 2 after administration of low dose cyclophosphamide. The trend was arrested, and the effect of C52 was completely abolished during the subsequent 8 to 9 days (13 to 22 days after tumor inoculation) (FIG. 12, B, shaded part). However, from day 22 to day 28 the tumor growth-promoting effect of C52 gradually appeared (FIG. 12, B). This is consistent with the known fact that the half-life of Treg in vivo is about 8 days, after which the depleted Treg will regenerate from its precursor (Vukmanovic-Stejic, et al. Human CD4+CD25hi Foxp3+ Regulatory T Cells are Derived by Rapid Turnover of Memory Populations in Vivo. J. Clin. Invest. 116:2423-2433, 2006). The mice were sacrificed on day 29 after tumor inoculation (i.e., day 16 after selective depletion of Tregs using LDCP), and CD4, CD25 and FoxP3 in spleens and lymph nodes were analyzed. The results showed that C52 significantly increased the regeneration of Treg in lymph nodes (FIG. 12, C), which is also consistent with the known fact that lymph nodes are the main peripheral tissue for the conversion of CD4+ naive T cells into Treg cells (Milanez-Almeida, et al. Foxp3+ Regulatory T-cell Homeostasis Quantitatively Differs in Murine Peripheral Lymph Nodes and Spleen. Eur. J. Immunol. 45:153-166, 2015).

Therefore, C52 can not only activate the function of Treg cells in vivo, but also promote the regeneration of Treg cells after their depletion.

### Example 12: Vimentin-Binding s-Triazine Derivatives, Such as C52, Increase the Ratio of Treg to CD4+ T Cells in Lymph Node and Reduce Symptoms and Tissue Damage in Mice with DSS-Induced Colitis In Vivo

The occurrence and progression of inflammatory bowel disease are closely related to vimentin (Mor-Vaknin, N. et al. Murine Colitis is Mediated by Vimentin. Sci Rep. 3:1045 2013), and the dysregulation of regulatory T cells plays a key role in it (Yamada, et al. Role of regulatory T cell in the pathogenesis of inflammatory bowel disease. World J Gastroenterol. 22:2195-2205, 2016). We used the DSS-induced colitis mouse model to demonstrate the activation of regulatory T cells and the therapeutic effect on autoimmune and inflammatory diseases of vimentin-bound s-triazine derivatives in vivo.

### Materials and Methods

Male BALB/c mice aged 6 to 7 weeks were given 2.8% DSS (Yisheng Biotechnology Co., Ltd., Shanghai) to induce ulcerative colitis through drinking water, and the model was established continuously for 7 days. From the first day of modeling, 3 mg/kg of C52 or 300 mg/kg of the positive drug mesalamine (5′-aminosalicylic acid, 5-ASA) was given by intragastric administration, and continued after the modeling 7 days. The model group was given the same volume of solvent every day. The normal control group was fed routinely without any treatment. From the first day of modeling, the animals in each group were weighed every day, and the health condition of the mice, appearance of feces and hematochezia were recorded. When the body weight of the mice lost more than 25% during the modeling process, the animals were euthanized in time. After 14 days of administration of C52, mice in each group were sacrificed by vertebral dislocation. The colon was harvested, photographed and its length measured. The colon near the rectum was cut and stored in 4% paraformaldehyde (Sevier Biotechnology Co., Ltd., Wuhan) for HE staining to observe the histopathological changes of the colon. Mesenteric lymph nodes were collected for flow cytometric detection of the proportion of Treg cells.

### Results and Conclusions

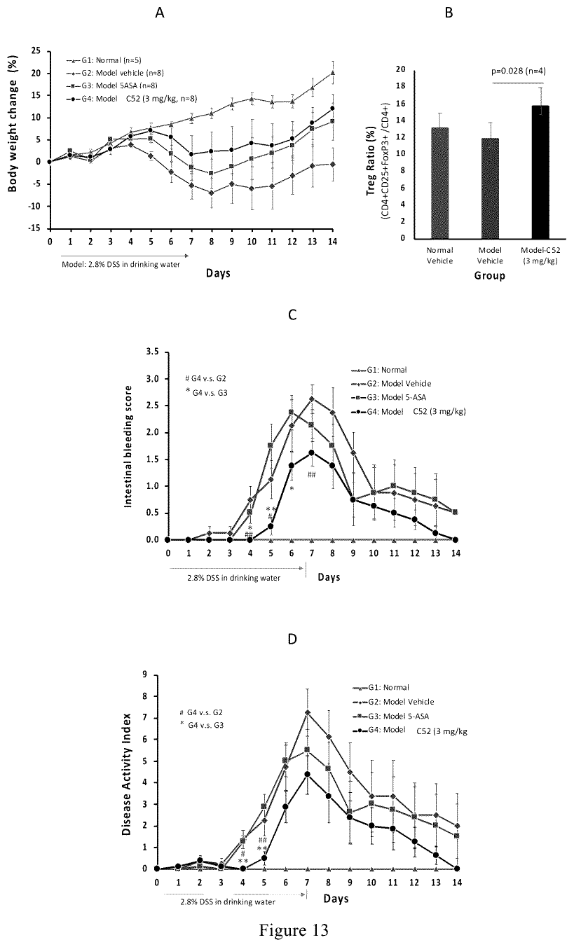

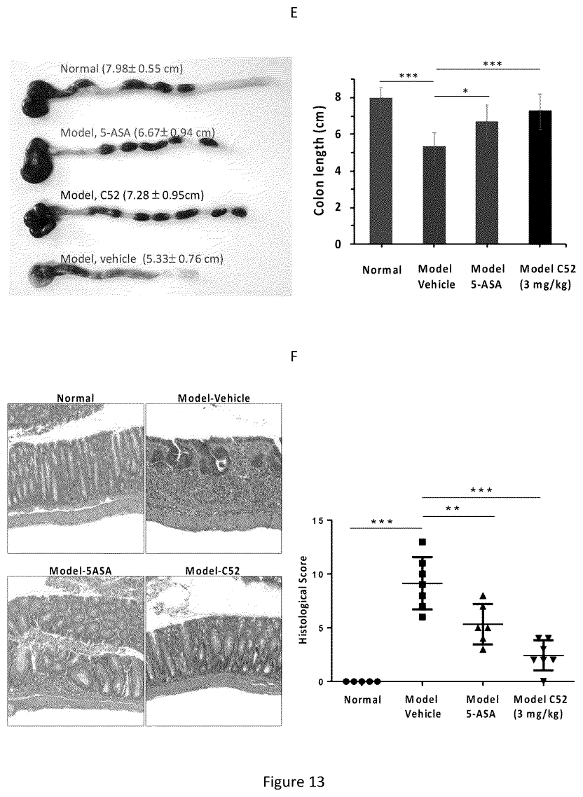

Vimentin is known to be associated with the pathogenesis of colitis, and Vim KO mice are protected from DSS-induced acute colitis (Mor-Vaknin, et al. Murine colitis is mediated by vimentin. Sci Rep. 3:1045, 2013). We speculate that the Treg function restrained by vimentin may be a mechanism for causing ulcerative colitis, and therefore, vimentin-targeting s-triazine derivatives should be effective in treating the disease. Indeed, mice with DSS-induced colitis showed significantly less weight loss (FIG. 13, A), reduced intestinal bleeding (FIG. 13, C) and decreased disease activity index (FIG. 13, D), there was also a marked improvement in shortened colon length (FIG. 13, E), and a very significant reduction in histological score (FIG. 13, F). The ratio of Treg to CD4+ T cells in the mesenteric lymph nodes was significantly higher in mice treated with compound C52 than in mice treated with vehicle control (FIG. 13, B).

Therefore, compound C52 increased the ratio of Tregs to CD4+ T cells in vivo and had a significant therapeutic effect on DSS-induced colitis in mice.

### Example 13: Vimentin-Binding s-Triazine Derivatives, Such as C52, can Effectively Treat MOG-Induced Experimental Autoimmune Encephalomyelitis (EAE) in Mice In Vivo

To further explore the potential application of immunomodulatory effects associated with vimentin targeting, the EAE mouse model of multiple sclerosis, a model of typical T cell-mediated autoimmune diseases, was used.

### Materials and Methods

The EAE model was induced by immunizing 40 female C57BL/6 mice with MOG 35-55 (myelin oligodendrocyte glycoprotein 35-55 peptide) in complete Freund's adjuvant. The 5 groups of animals included 3 groups of C52-treated animals, namely 100 mg/kg (G6, n=8), 30 mg/kg (G5, n=8) or 10 mg/kg (G4, n=8), and 1 group of positive drug FTY720 (1 mg/kg) treated animals (G3, n=8), and a group of vehicle control (G2, n=8), orally administered to mice once a day from day 0 to day 30. Four normal mice (G1, n=4) were used as normal controls. EAE severity was assessed by clinically scoring mice once a day from day 0 to day 30 after immunization. The maximum disease score, mean days to onset of clinical symptoms, EAE duration, AUC score, suppression rate and mortality were determined.

### Reagent Preparation:

MOG solution: Dissolve MOG 35-55 in saline to a concentration of 2 mg/mL.
Modified complete Freund's adjuvant (CFA): Add heat-killed Mycobacterium tuberculosis to make complete Freund's adjuvant at a final concentration of 4 mg/mL.
Emulsion preparation: Emulsify an equal volume of 4 mg/mL CFA in a 2 mg/mL solution of MOG 35-55 using a high-speed homogenizer at 30,000 rpm for 1.5 h on ice.
Induction of EAE: Mice were anesthetized with isoflurane, and then 100 μL of total emulsion was injected subcutaneously into three sites on the shaved back of the mice to induce EAE. The three locations are at the midline of the back between the shoulders, and on both sides of the lower end of the midline of the back. This day is recorded as day 0. Animals in all immunization groups were intraperitoneally injected with Bordetella pertussis toxin (PTX, 200 ng dissolved in 200 μL PBS) on the day of immunization and within 48 h afterward.

### EAE Clinical Scoring:

| Score | Clinical symptom |
|---|---|
| 0 | Norma, no sign of disease |
| 1 | Weakness of tail or hindlimbs, but not both |
| 2 | Weakness of tail and hindlimbs |
| 3 | Partial paralysis of the hind limbs |
| 4 | Full paralysis of the hind limbs |
| 5 | Imminent death from EAE, or euthanasia |

### Results and Conclusions

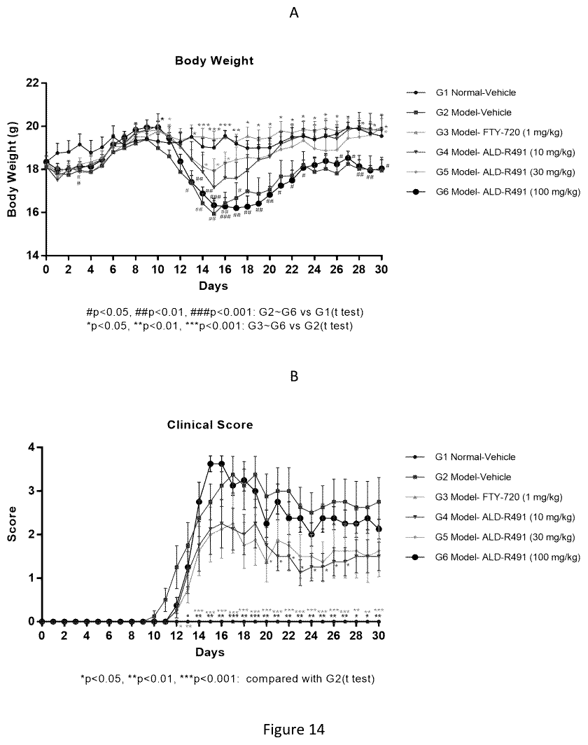

Both low and medium doses of C52-treated mice had less weight loss (FIG. 14, A), relatively delayed disease onset, and lower disease scores compared to vehicle control-treated mice (FIG. 14, B). However, high-dose C52 treatment showed no therapeutic effect (FIG. 14). Vimentin is known to regulate Treg and NLRP3 inflammasomes, and exosomes can mediate activation or suppression of immunity (Lindenbergh, et al. Antigen presentation by extracellular vesicles from professional antigen-presenting cells. Annu. Rev. Immunol. 36:435-459, 2018; Anel, et al. Role of exosomes in the regulation of T-cell mediated immune responses and in autoimmune disease. Cells 8:154, 2019). As mesenchymal cells, all immune cells express a large amount of vimentin and actively release exosomes. Certain immune cell subsets, such as Tregs, MDSCs, and M2 macrophages, or CD8+ T cells, NK cells, and dendritic cells, are known to have pro- or anti-cancer effects. Compound C52 combined with vimentin can block exosomes, and its overall effect on the immune system in vivo will depend on the net balance of the compound's effect on these various cells. Due to different concentrations, the effect of compound C52 on the immune system can be inhibitory or stimulatory.

Vimentin-binding s-triazine derivative C52 can effectively treat T cell-mediated autoimmune diseases by activating Treg cells in vivo at low doses. However, due to the effect of this compound on various types of immune cells at high doses, it is likely to counteract its activation effect on Treg, which may diminish the effect of the compound in treating EAE. In conclusion, the use of vimentin-binding s-triazine derivatives in the treatment of immune-related diseases has been validated in one more autoimmune disease model.

It should be understood that the foregoing description describes preferred embodiments of the invention and that modifications may be made thereto without departing from the spirit or scope of the invention as set forth in the claims. To particularly point out and distinctly claim the subject matter, which is regarded as invention, the following claims conclude this specification.

1. A method for treating or preventing diseases involving cell endocytosis, exocytosis and endosomal trafficking, or for treating or preventing diseases involving a deficiency of regulatory T in quantity and/or function of regulating T cells, comprising administering to a subject in need thereof a s-triazine derivative shown in the following formula A, or its pharmaceutically acceptable carrier, prodrug, enantiomer, diastereoisomer, tautomer or solvate: 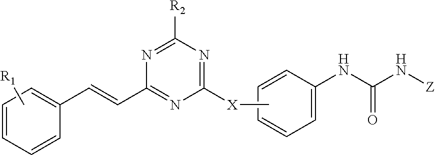
(A)
wherein:
R₁ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl or aminomethyl;
R₂ is —NR₄R₅, wherein R₄ and R₅ are independently selected from a group consisting of hydrogen, C₁-C₆ alkyl, and C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, hydroxyl, C₁-C₆ alkyl, or C₁-C₆ haloalkyl;
Z is aryl or heteroaryl optionally substituted by 1-3 R₃;
R₃ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl, aminomethyl or —CORₐ;
wherein Rₐ is OH or NR₇R₈, wherein R₇ and R₈ are independently selected from hydrogen, C₁-C₆ alkyl optionally substituted with one or more substituents selected from halogen or NR₉R₁₀, and C₁-C₆ alkyl substituted with 3-(C₂-C₆ alkynyl)-3H-diaziridinyl; or R₇, R₈ and the nitrogen atom bonded to them form a 4- to 6-membered heterocyclic ring containing optionally an additional heteroatom selected from N, O and S, and optionally substituted with C₁-C₆ alkyl;
wherein R₉ and R₁₀ are independently selected from hydrogen and C₁-C₆ alkyl; or R₉, R₁₀ and the nitrogen atom bonded to them form a 4- to 6-membered heterocyclic ring containing optionally an additional heteroatom selected from N, O and S; and
X is NH or O, linked to the phenyl group at a para- or meta-position.

2. The method according to claim 1, wherein the compound of formula A has a structure shown in formula I below: 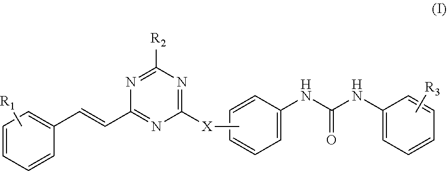
wherein:
R₁ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl or aminomethyl;
R₂ is —NR₄R₅, wherein R₄ and R₅ are independently selected from a group consisting of hydrogen, C₁-C₆ alkyl, and C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, hydroxyl, C₁-C₆ alkyl, or C₁-C₆ haloalkyl;
R₃ is hydrogen, halogen, nitro, amino, hydroxyl, C₁-C₁₂ alkyl, C₁-C₆ alkoxyl, C₁-C₆ alkylamino, di-C₁-C₆ alkylamino, hydroxymethyl, aminomethyl or —CORₐ;
wherein Rₐ is OH or NR₇R₈, wherein R₇ and R₈ are independently selected from hydrogen, C₁-C₆ alkyl optionally substituted with one or more substituents selected from halogen or NR₉R₁₀, and C₁-C₆ alkyl substituted with 3-(C₂-C₆ alkynyl)-3H-diaziridinyl; or R₇, R₈ and the nitrogen atom bonded to them form a 4- to 6-membered heterocyclic ring containing optionally an additional heteroatom selected from N, O and S, and optionally substituted with C₁-C₆ alkyl;
wherein R₉ and R₁₀ are independently selected from hydrogen and C₁-C₆ alkyl; or R₉, R₁₀ and the nitrogen atom bonded to them form a 4- to 6-membered heterocyclic ring containing optionally an additional heteroatom selected from N, O and S; and
X is NH or O, linked to the phenyl group at a para- or meta-position.

3. The method according to claim 2, wherein:
R₁ is hydrogen, halogen or nitro; and/or
R₂ is —NR₄R₅, wherein R₄ and R₅ are independently selected from hydrogen, C₁-C₆ alkyl, and C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, hydroxyl or C₁-C₆ alkyl; and/or
R₃ is hydrogen, halogen, nitro, amino, hydroxyl, C₁-C₆ alkyl, hydroxymethyl, aminomethyl or —CORₐ, wherein, Rₐ is OH or NR₇R₈, wherein R₇ and R₈ are independently selected from hydrogen, C₁-C₆ alkyl optionally substituted with one or more substituents selected from halogen or NR₉R₁₀, and C₁-C₆ alkyl substituted with 3-(C₂-C₆ alkynyl)-3H-diaziridinyl; or R₇, R₈ and the nitrogen atom bonded to them form a 4- to 6-membered heterocyclic ring containing optionally an additional heteroatom selected from N, O and S; R₉ and R₁₀ are independently selected from hydrogen and C₁-C₆ alkyl, or R₉, R₁₀ and the nitrogen atom bonded to them form a 4 to 6-membered heterocyclic ring optionally containing additional heteroatoms selected from N, O, S; and/or
X is NH, linked to the phenyl group at a para- or meta-position, or X is O, linked to the phenyl group at a para position.

4. The method according to claim 2, wherein in formula I:
R₁ is hydrogen, halogen or nitro;
R₂ is —NR₄R₅, wherein R₄ and R₅ are independently selected from hydrogen, C₁-C₆ alkyl, C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, hydroxyl or C₁-C₆ alkyl; and
R₃ is hydrogen, halogen, nitro, amino, hydroxyl, C₁-C₆ alkyl, hydroxymethyl, aminomethyl or —CORₐ; wherein Rₐ is OH or NR₇R₈, wherein R₇ and R₈ are independently selected from hydrogen, C₁-C₆ alkyl optionally substituted with one or more substituents selected from halogen or NR₉R₁₀, and C₁-C₆ alkyl substituted with 3-(C₂-C₆ alkynyl)-3H-diaziridinyl; or R₇, R₈ and the nitrogen atom bonded to them form a 4- to 6-membered heterocyclic ring containing optionally an additional heteroatom selected from N, O and S, and optionally substituted with C₁-C₆ alkyl; wherein R₉ and R₁₀ are independently selected from hydrogen and C₁-C₆ alkyl; or R₉, R₁₀ and the nitrogen atom bonded to them form a 4- to 6-membered heterocyclic ring containing optionally an additional heteroatom selected from N, O and S; or
in formula I:
R₁ is hydrogen, halogen or nitro;
R₂ is —NR₄R₅, wherein R₄, R₅ and the nitrogen atom bonded to them form a saturated 4- to 6-membered heterocyclic ring containing optionally an additional heteroatom selected from NR₆ and O, wherein the heterocyclic ring is optionally substituted with hydroxyl or C₁-C₆ alkyl, and wherein R₆ is hydrogen, hydroxyl or C₁-C₆ alkyl;
R₃ is halogen or CORₐ, wherein Rₐ is OH or NR₇R₈, wherein R₇ and R₈ are independently selected from C₁-C₆ alkyl optionally substituted by NR₉R₁₀ and C₁-C₆ alkyl optionally substituted by 3-(C₂-C₆ alkynyl)-3H-diaziridinyl, or R₇, R₈ and the nitrogen atom bonded to them form a saturated 4- to 6-membered heterocyclic ring containing optionally an additional heteroatom selected from N or O and optionally substituted by C₁-C₆ alkyl; wherein R₉ and R₁₀ are independently selected from hydrogen and C₁-C₆ alkyl; or R₉, R₁₀ and the nitrogen atom bonded to them form a saturated 4- to 6-membered heterocyclic ring containing optionally an additional heteroatom selected from N or O; and
X is NH, linked to phenyl group at para or meta position; or
in formula I:
R₁ is hydrogen, halogen or nitro;
R₂ is —NR₄R₅, wherein R₄ and R₅ are independently selected from hydrogen, C₁-C₆ alkyl, C₁-C₆ haloalkyl; or R₄, R₅ and the nitrogen atom bonded to them form a saturated or unsaturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, hydroxyl or C₁-C₆ alkyl; and
R₃ is hydrogen, halogen, nitro, amino, hydroxyl, C₁-C₆ alkyl, hydroxymethyl, aminomethyl or —CONR₇R₈; wherein R₇, R₈ are independently selected from hydrogen, C₁-C₆ optionally substituted alkyl, or R₇, R₈ and the nitrogen atom bonded to them form a 4 to 6-membered heterocyclic ring containing optionally an additional heteroatom selected from N, O, S, wherein C₁-C₆ alkyl can be optionally substituted by one or more halogens, C₁-C₆ alkylamino, di-C₁-C₆ alkylamino;
the X group is NH or O at meta and para position; or
in formula I:
R₁ is hydrogen, halogen or nitro;
R₂ is —NR₄R₅, wherein R₄, R₅ are independently selected from hydrogen, C₁-C₆ alkyl, or R₄, R₅ and the nitrogen atom bonded to them form a saturated 4 to 6-membered heterocyclic ring containing optionally an additional heteroatom selected from NR₆, O and S, wherein the heterocyclic ring may be substituted with hydroxyl, halogen, nitro, amino or C₁-C₆ alkyl, wherein R₆ is hydrogen, or C₁-C₆ alkyl; and
R₃ is hydrogen, halogen, or —CONR₇R₈; wherein R₇, R₈ are independently selected from hydrogen, C₁-C₆ optionally substituted alkyl, or R₇, R₈ and the nitrogen atom bonded to them form a 4 to 6-membered saturated heterocyclic ring containing optionally an additional heteroatom selected from N, O, S, wherein C₁-C₆ alkyl can be optionally substituted by one or more C₁-C₆ alkylamino, di-C₁-C₆ alkylamino;
the X group is NH or O at meta and para position.

5. The method according to claim 2, wherein the compound of formula I has a structure shown in the following formula I-1 or the following formula I-2: 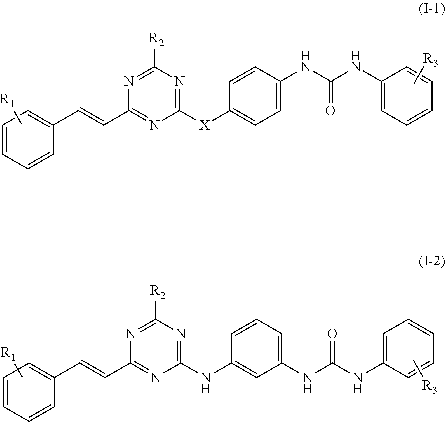
wherein:
R₁ is selected from H, halogen and nitro;
R₂ is selected from morpholinyl, pyrrolidinyl, piperazinyl and azetidinyl optionally substituted by hydroxyl or C₁-C₆ alkyl; and
R₃ is halogen or CORₐ; wherein, Rₐ is OH or NR₇R₈, wherein R₇ and R₈ are independently selected from C₁-C₆ alkyl optionally substituted by NR₉R₁₀ and C₁-C₆ alkyl optionally substituted by 3-(C₂-C₆ alkynyl)-3H-diaziridinyl, or R₇, R₈ and the nitrogen atom bonded to them form a 4 to 6-membered saturated heterocyclic ring containing optionally an additional heteroatom selected from N and O, and optionally substituted with C₁-C₆ alkyl optionally containing additional heteroatoms selected from N or O Heterocycle; R₉ and R₁₀ are independently selected from hydrogen and C₁-C₆ alkyl, or R₉, R₁₀ and the nitrogen atom bonded to them form a 4 to 6-membered saturated heterocyclic ring containing optionally an additional heteroatom selected from N and O.

6. The method according to claim 2, wherein the compound described in formula I has a structure shown in following formula I-3: 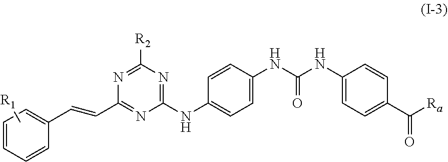
wherein:
R₁ is H;
R₂ is morpholinyl;
Rₐ is OH or NR₇R₈, wherein R₇, R₈ are independently selected from C₁-C₆ alkyl optionally substituted by NR₉R₁₀ and C₁-C₆ alkyl substituted by 3-(C₂-C₆ alkynyl)-3H-diaziridinyl, or R₇, R₈ and the nitrogen atom bonded to them form a 4 to 6-membered saturated heterocyclic ring optionally substituted by C₁-C₆ alkyl and optionally containing additional heteroatoms selected from N and O; R₉ and R₁₀ are independently selected from hydrogen and C₁-C₆ alkyl, or R₉, R₁₀ and the nitrogen atom bonded to them form a 4 to 6-membered saturated heterocyclic optionally containing additional heteroatoms selected from N and O.

7. The method according to claim 1, wherein the compound described in formula A has a structure shown in following formula A-1: 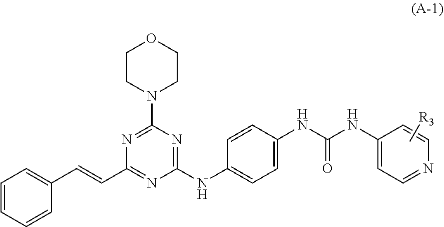
wherein:
R₃ is hydrogen, halogen, nitro, amino, hydroxyl, C1-C6 alkyl, hydroxymethyl, aminomethyl or —CONR₇R₈; wherein R₇, R₈ are independently selected from hydrogen, C₁-C₆ optionally substituted alkyl, or R₇, R₈ and the nitrogen atom bonded to them form a 4 to 6-membered heterocyclic ring containing optionally an additional heteroatom selected from N, O, S, wherein C₁-C₆ alkyl can be optionally substituted by one or more halogens, C₁-C₆ alkylamino, di-C₁-C₆ alkylamino.

8. The method according to claim 1, wherein the compound of formula A is selected from the following compounds and their pharmaceutically acceptable salts, prodrugs, enantiomers, diastereoisomers, tautomers and solvates:
(1) compounds L1-L22 represented by the following structural formula:

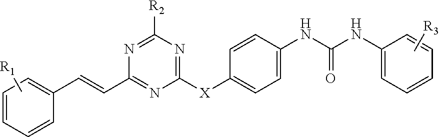

| ID number | R₁ | R₂ | R₃ |
|---|---|---|---|
| L1 | H | 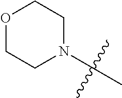 [SMILES: *N1CCOCC1] | 4-Cl |
| L2 | H | 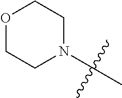 [SMILES: *N1CCOCC1] | 4-F |
| L3 | H | 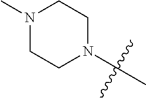 [SMILES: *N1CCN(C)CC1] | 4-Cl |
| L4 | H | 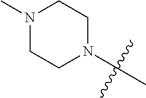 [SMILES: *N1CCN(C)CC1] | 4-F |
| L5 | H | 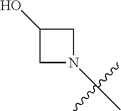 [SMILES: *N1CC(O)C1] | 4-Cl |
| L6 | 3-Cl | 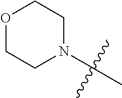 [SMILES: *N1CCOCC1] | 4-Cl |
| L7 | 3-Cl | 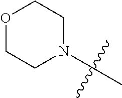 [SMILES: *N1CCOCC1] | 4-F |
| L8 | 3-Cl | 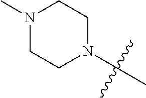 [SMILES: *N1CCN(C)CC1] | 4-Cl |
| L9 | 3-Cl | 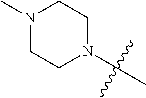 [SMILES: *N1CCN(C)CC1] | 4-F |
| L10 | 4-Cl | 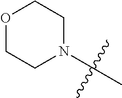 [SMILES: *N1CCOCC1] | 4-Cl |
| L11 | 4-Cl | 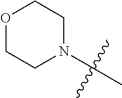 [SMILES: *N1CCOCC1] | 4-F |
| L12 | 4-Cl | 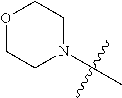 [SMILES: *N1CCOCC1] | 3-F |
| L13 | 4-Cl | 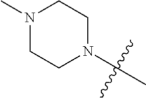 [SMILES: *N1CCN(C)CC1] | 4-Cl |
| L14 | 4-Cl | 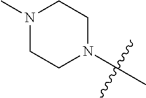 [SMILES: *N1CCN(C)CC1] | 4-F |
| L15 | 4-Cl | 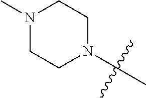 [SMILES: *N1CCN(C)CC1] | 3-F |
| L16 | 3-NO₂ | 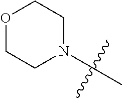 [SMILES: *N1CCOCC1] | 4-Cl |
| L17 | 3-NO₂ | 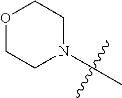 [SMILES: *N1CCOCC1] | 4-F |
| L18 | 3-NO₂ | 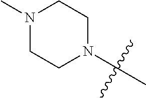 [SMILES: *N1CCN(C)CC1] | 4-Cl |
| L19 | 3-NO₂ | 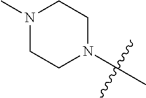 [SMILES: *N1CCN(C)CC1] | 4-F |
| L20 | 3-NO₂ | 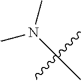 [SMILES: *N(C)C] | 4-Cl |
| L21 | 3-NO₂ | 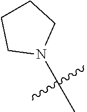 [SMILES: *N1CCCC1] | 4-Cl |
| L22 | 3-NO₂ | 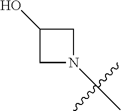 [SMILES: *N1CC(O)C1] | 4-Cl |

(2) compounds L233L28 represented by the following structural formula:

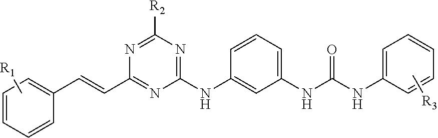

| ID number | R₁ | R₂ | R₃ |
|---|---|---|---|
| L23 | H | 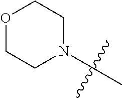 [SMILES: *N1CCOCC1] | 4-Cl |
| L24 | H | 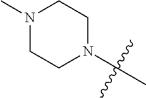 [SMILES: *N1CCN(C)CC1] | 4-Cl |
| L25 | 3-Cl | 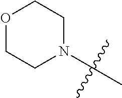 [SMILES: *N1CCOCC1] | 4-Cl |
| L26 | 3-Cl | 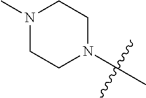 [SMILES: *N1CCN(C)CC1] | 4-Cl |
| L27 | 3-NO₂ | 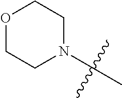 [SMILES: *N1CCOCC1] | 4-Cl |
| L28 | 3-NO₂ | 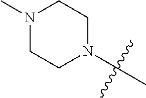 [SMILES: *N1CCN(C)CC1] | 4-Cl |

(3) compounds L29-L38 represented by the following structural formula:

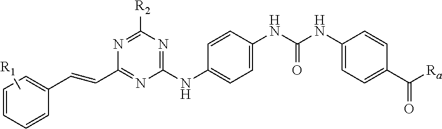

| ID number | R₁ | R₂ | Rₐ |
|---|---|---|---|
| L29 | H | 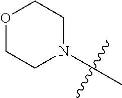 [SMILES: *N1CCOCC1] | 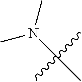 [SMILES: *N(C)C] |
| L30 | H | 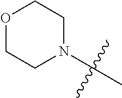 [SMILES: *N1CCOCC1] | 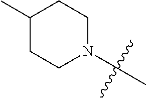 [SMILES: *N1CCC(C)CC1] |
| L31 | H | 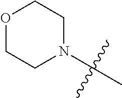 [SMILES: *N1CCOCC1] | 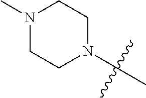 [SMILES: *N1CCN(C)CC1] |
| L32 | H | 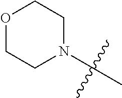 [SMILES: *N1CCOCC1] | 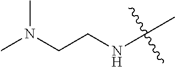 [SMILES: *NCCN(C)C] |
| L33 | H | 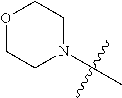 [SMILES: *N1CCOCC1] | 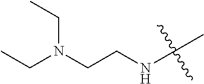 [SMILES: *NCCN(CC)CC] |
| L34 | H | 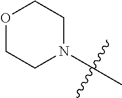 [SMILES: *N1CCOCC1] | 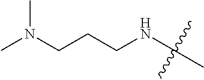 [SMILES: *NCCCN(C)C] |
| L35 | H | 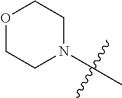 [SMILES: *N1CCOCC1] | 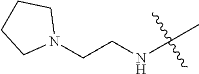 [SMILES: *NCCN1CCCC1] |
| L36 | H | 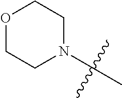 [SMILES: *N1CCOCC1] | 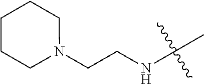 [SMILES: *NCCN1CCCCC1] |
| L37 | H | 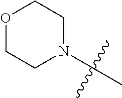 [SMILES: *N1CCOCC1] | 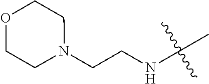 [SMILES: *NCCN1CCOCC1] |
| L38 | H | 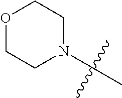 [SMILES: *N1CCOCC1] | OH |

(4) compounds L39-L41 represented by the following structural formula:

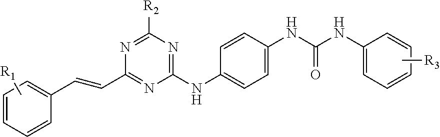

| ID number | R₁ | R₂ | R₃ |
|---|---|---|---|
| L39 | H | 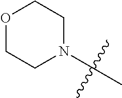 [SMILES: *N1CCOCC1] | 4-OCH₃ |
| L40 | H | 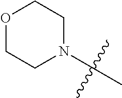 [SMILES: *N1CCOCC1] | 4-Cl |
| L41 | H | 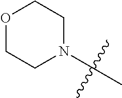 [SMILES: *N1CCOCC1] | 4-F |

and (5) compound L42:

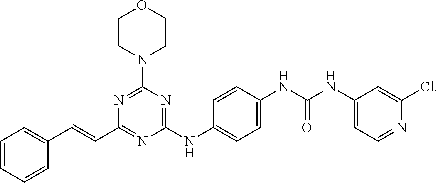 [SMILES: O=C(Nc1ccc(Nc2nc(/C=C/c3ccccc3)nc(N3CCOCC3)n2)cc1)Nc1ccnc(Cl)c1]

9. The method according to claim 1, wherein:
the diseases associated with cellular processes involving endocytosis, exocytosis, and endosomal trafficking are vimentin-mediated diseases, including cancer, pathogen infections, and other diseases characterized by abnormalities in one or more cellular processes;
the diseases related to the inadequate number and/or function of regulatory T cells are autoimmune diseases and inflammatory diseases.

10. The method according to claim 9, wherein:
the cancer has the following characteristics: its cancer cells use vimentin to achieve invasive growth, use endocytosis to uptake nutrients, use exocytosis to release exosomes as a mediator to communicate with other cells, and create a microenvironment suitable for cancer cell growth and metastasis;
the pathogen of interest encompasses bacteria and/or viruses that enter host cells via endocytosis, traverse within the cell through the endosomal pathway, and/or release their progeny via the exosomal pathway;
the diseases associated with inadequate regulatory T cell quantity and/or function include conditions characterized by cytokine storm-induced ailments, such as acute respiratory distress syndrome and organ failure. Additionally, they include diseases featuring an inflammatory component, such as post-cancer chemotherapy injury, infectious diseases, and Alzheimer's disease.

11. The method according to claim 1, wherein Z is aryl or heteroaryl optionally substituted by 1-3 R₃, wherein said aryl is 6-14 membered aryl; said heteroaryl is 5- to 10-member heteroaryl.

12. The method according to claim 11, wherein the 6-14 membered aryl is phenyl or naphthyl; said heteroaryl is 5- to 10-member heteroaryl is nitrogen-containing heteroaryl selected from a group consisting of imidazolyl, pyrazolyl, pyridyl, pyrimidinyl, pyrazinyl, pyridazinyl, pyrrolyl, triazolyl and tetrazolyl.

13. The method according to claim 1, wherein Z is phenyl or pyridyl optionally substituted by 1 or 2 R₃.

14. The method according to claim 3, wherein:
R₁ is H, F, Cl or nitro; and/or
R₄, R₅ are independently selected from hydrogen, C₁-C₆ alkyl, or R₄, R₅ and the nitrogen atom bonded to them form a saturated 4- to 6-membered heterocyclic ring containing optionally an additional heteroatom selected from NR₆, O and S, wherein the heterocyclic ring may be substituted with hydroxyl, halogen, nitro, amino or C₁-C₆ alkyl; wherein R₆ is hydrogen or C₁-C₆ alkyl; or, R₄ and R₅ and the nitrogen atom bonded to them form a saturated 4- to 6-membered heterocyclic ring containing optionally an additional heteroatom selected from NR₆ and O, wherein the heterocyclic ring is optionally substituted by a substituent selected from hydroxyl and C₁-C₆ alkyl, wherein, R₆ is hydrogen or C₁-C₆ alkyl; and/or
R₃ is halogen, C₁-C₆ alkoxy or —CORₐ, wherein Rₐ is OH or NR₇R₈, wherein R₇ and R₈ are independently selected from C₁-C₆ alkyl optionally substituted by NR₉R₁₀ and C₁-C₆ alkyl optionally substituted by 3-(C₂-C₆ alkynyl)-3H-diaziridinyl, or R₇, R₈ and the nitrogen atom bonded to them form a 4- to 6-membered saturated heterocyclic ring optionally substituted C₁-C₆ alkyl optionally containing additional heteroatoms selected from N or O, wherein R₉ and R₁₀ are independently selected from hydrogen and C₁-C₆ alkyl, or R₉, R₁₀ and the nitrogen atom bonded to them form a 4 to 6-membered saturated heterocyclic ring optionally containing additional heteroatoms selected from N or O.

15. The method according to claim 3, wherein:
R₄ and R₅ and the nitrogen atom bonded to them form a saturated 4- to 6-membered heterocyclic ring selected from morpholinyl, pyrrolidinyl, piperazinyl, piperidinyl and azetidinyl; and/or
the 4 to 6-membered heterocyclic ring formed by R₇, R₈ and the nitrogen atom bonded to them and the 4 to 6-membered heterocyclic ring formed by R₉, R₁₀ and the nitrogen atom bonded to them are piperidinyl, piperazinyl, pyrrolidinyl or morpholinyl; and/or,
when R₃ is a non-H substituent, it is located at the meta or para position of the phenyl group.

16. The method according to claim 5, wherein:
in formula I-2, R₃ is halogen;
in formula I-1, R₁ is selected from H and halogen; R₂ is morpholino; R₃ is halogen or CORₐ, wherein Rₐ is OH or NR₇R₈, wherein R₇, R₈ and the nitrogen atom bonded to them form a 4 to 6-membered saturated heterocyclic ring optionally substituted by C₁-C₆ alkyl optionally containing additional heteroatoms selected from N and O.

17. The method according to claim 16, wherein:
in formula I-1, R₁ is Cl; R₂ is morpholinyl; R₃ is halogen or CORₐ, wherein Rₐ is OH or NR₇R₈, wherein R₇, R₈ and the nitrogen atom bonded to them form piperidinyl, piperazinyl, pyrrole alkyl or azetidinyl optionally substituted by C₁-C₆ alkyl.

18. The method according to claim 7, wherein R₃ is H or halogen.

19. The method according to claim 9, wherein the diseases related to the inadequate number and/or function of regulatory T cells include: inflammatory bowel disease (IBD), multiple sclerosis (MS), SARS-CoV infection (e.g. COVID-19), systemic lupus erythematosus (SLE), type 1 diabetes (T1D), psoriasis, graft-versus-host disease (GvHD), myasthenia gravis (MG), arthritis, scleroderma, dermatomyositis, vasculitis, neuritis, autoimmune hemolytic anemia, pernicious anemia with chronic atrophic gastritis, pulmonary hemorrhagic nephritic syndrome, primary biliary cirrhosis, thyroid autoimmune disease, pemphigus, Sjorgen's syndrome, grapevine meningitis, allergic conjunctivitis, celiac disease, nonspecific colitis, fibrosis, autoimmune encephalomyelitis (EAE), atherosclerosis, chronic kidney disease, osteoporosis, allergies, fibromyalgia and neurodegeneration.

20. The method according to claim 10, wherein
the cancers include: colon cancer, pancreatic cancer, ovarian cancer, gastric cancer, breast cancer, thyroid cancer, liver cancer, kidney cancer, lung cancer, prostate cancer, sarcoma, glioma, leukemia and multiple myeloma;
the pathogen is selected from a range of options including Coronavirus (including SARS-CoV-2), HIV, Influenza virus, Hepatitis B virus, Hepatitis C virus, Human papillomavirus, Ebola virus, Dengue virus, Escherichia coli, Salmonella enteritidis, Anaplasma phagocytophilum, Chlamydia trachomatis, Streptococcus pyogenes, Mycobacterium tuberculosis, Mycobacterium avium, and Propionibacterium acnes;
the infection caused by the pathogens from one or more of the described pathogens, and the resulting disease falls under the category of communicable or infectious diseases, including but not limited to COVID-19, AIDS, hepatitis B, influenza, and adherent invasive Escherichia coli (AIEC) infection.
